(19) United States
(12) Patent Application Publication
(10) Pub. No.: US 2022/0378744 A1
(43) Pub. Date: Dec. 1, 2022

(54) COMPOUNDS FOR TREATING PROLIFERATOR-ACTIVATED RECEPTORS (PPAR) MEDIATED DISEASES OR CONDITIONS

(71) Applicant: MEDIPURE PHARMACEUTICALS INC., North Vancouver (CA)

(72) Inventors: Safwat M. RABEA, North Vancouver (CA); Kaiji HU, North Vancouver (CA); Rakshit D. KODEKALRA, North Vancouver (CA); K.K. Durgaro VISHWANADHAM, North Vancouver (CA); Rashmi TIWARI-PANDEY, North Vancouver (CA); Nihar PANDEY, North Vancouver (CA)

(21) Appl. No.: 17/727,125

(22) Filed: Apr. 22, 2022

Related U.S. Application Data
(60) Provisional application No. 63/179,075, filed on Apr. 23, 2021.

(51) Int. Cl. A61K 31/416; A61K 31/05; A61P 37/06; C07D 231/56; C07D 403/12; C07D 401/12; A61K 31/4439

(52) U.S. Cl. CPC A61K 31/416; A61K 31/05; A61P 37/06; C07D 231/56; C07D 403/12; C07D 401/12; A61K 31/4439

(57) ABSTRACT

Novel compounds, in particular novel peroxisome proliferator-activated receptor (PPAR) agonist compounds, are provided. Pharmaceutical formulations for treating PPAR mediated disorder or condition are also provided. The formulations may be a combination of cannabinoids and the PPAR agonist compounds. The PPAR mediated disorder or condition may include inflammation related diseases such as psoriasis, psoriatic arthritis and atopic dermatitis.

### PRIORITY

This application claims the benefit of U.S. provisional application 63/179,075 filed Apr. 23, 2021.

### FIELD OF INVENTION

The present disclosure relates to novel compounds, in particular that activate peroxisome proliferator-activated receptors (PPARs). This disclosure further relates to the methods and uses of the novel compounds.

### BACKGROUND OF THE INVENTION

Inflammation plays a significant role in host defenses against infectious agents and injury, initiating the intracellular signaling pathways, thus activates the production of inflammatory mediators. Primary inflammatory stimuli, including microbial products and cytokines such as interleukin-1β (IL-1β), interleukin-6 (IL-6), and tumor necrosis factor-α (TNF-α), mediate inflammation through interaction with the Toll-like receptors (TLRs), IL-1 receptor (IL-1R), IL-6 receptor (IL-6R), and the TNF receptor (TNFR) (Manley et al, 2005) Cytokines are involved in both inflammation and anti-inflammatory activities via multifunctional molecules depending on the manner of the inflammatory response (Chousterman et al., 2005). The persistence of irregular inflammatory responses gradually results in chronic systemic inflammatory changes thus aggravating tissue injury, contributing to the pathophysiology of various chronic diseases, such as cancer, obesity, ischemic heart disease, stroke, diabetes mellitus, chronic kidney disease, liver diseases, skin-related disorders and autoimmune and neurodegenerative conditions (Miller et al, 2011). Systemic Chronic Inflammation (SCI) is associated with increased risk for developing a variety of chronic inflammatory diseases significantly causing morbidity and mortality worldwide, with more than 50% of all deaths being attributable to inflammation-related diseases (Furman et al., 2019).

Inflammation predisposes to the development of cancer and promotes all stages of tumorigenesis (Greten & Grivennikov, 2019). Inflammatory mediators, including cytokines like TNF, IL-1, and IL-6, growth factors, chemokines, and proteases produced by tumor-associated lymphocytes and macrophages can enhance tumor cell growth and metastasis. Tumor-associated macrophages release inflammatory mediators that stimulate tumor angiogenesis and lymphangiogenesis (Zumsteg & Christofori, 2009), and produce cytokines, including transforming growth factor (TGF) β and IL-10, that can directly suppress immune responses (Zamarron & Chen, 2011).

Inflammation plays a potential role in the pathogenesis of a range of metabolic disorders, such as insulin resistance, diabetes mellitus, nonalcoholic fatty liver disease (NAFLD), and cardiovascular disease (CVD). The overlap between inflammatory and metabolic sensors and their downstream tissue responses indicates that inflammation plays a crucial role in the numerous complications of obesity (Fuentes et al., 2013).

Psoriasis and NAFLD are part of an intriguing network of genetic, clinical, and pathophysiological features, and share multiple inflammatory and cytokine-mediated mechanisms. It can be anticipated that mechanisms underlying the association between NAFLD and psoriasis are multifactorial (involving both genetic and environmental factors) and often overlap with metabolic abnormalities, which frequently coexist in psoriatic patients. Pro-inflammatory adipokines such as IL-6, leptin, and resistin seem to play a role in the pathogenesis of both NAFLD and psoriasis (Prussick et al, 2015).

Skin disorders are common. Many people are affected by proliferative and/or inflammatory skin disorders that result in unsightly and painful rashes, papules, eczema, acne, and erythematous plaques. Inflammatory skin disorders often result in intense psychosocial distress and have been linked to the precipitation of depression in many patient populations. About 2-3% of the worldwide population i. e. about 125 million people have psoriasis, according to the World Psoriasis Day consortium, and over 8 million people in the United States alone (Nestle et al., 2009).

Psoriasis as one of the inflammatory skin disorders, is an immune-mediated (autoimmune) chronic inflammatory skin disorder. It is characterized by hyperproliferation and abnormal differentiation of the keratinocytes as well as infiltration of immune cells into both the dermis and the epidermis (Nestle et al., 2009). Psoriasis is known to be the result of a complex interplay between genetics, environmental triggers, and the immune system (Nestle et al., 2009). Psoriasis may appear at any age, however, mostly (˜75%) observed before the age of 40 (type 1 or early-onset psoriasis) (Laws & Young, 2010). Though psoriasis can appear on any location in the body, it generally affects the outside region of the elbows, knees, palms, feet, body folds, chest and back. Psoriasis typically affects the skin, but may also affect the joints, and has been associated with psoriatic arthritis.

The symptoms of psoriasis are exacerbated by many factors including psychological stress, smoking, certain drugs, skin injury, obesity, alcoholic beverages, streptococcal infections of the throat and HIV.

There are several types of psoriasis including plaque, guttate, inverse, pustular and erythrodermic. Psoriasis has a large spectrum of clinical features and evolution, so no complete agreement on the classification of the clinical variants exists. However, the most common forms that are known include: (I) Plaque Psoriasis: the commonest form affecting more than 80% of affected patients and characterized by dry scaly patches, (II) Inverse Psoriasis: localized in the skin folds and characterized by smooth inflamed lesions, (III) Erythrodermic Psoriasis: characterized by exfoliation of fine scaly skin with pain and itching, (IV) Guttate Psoriasis: characterized by drop-like dots, erythematous-to-salmon-pink papules, usually with a fine scale, (V) Pustular Psoriasis: contains pus-filled blisters or pustules, (VI) Others including scalp psoriasis and nail psoriasis.

Among them, the most common clinical variant is plaque-type of psoriasis and appears as raised, round or oval red patches covered with silvery white buildup of dead skin cells. Furthermore, it is characterized by erythematous scaly plaques, variable in size, frequently located in lower back, scalp, knees, elbows and intergluteal cleft (Saraceno et al., 2009). They are often painful, itchy and can easily crack and bleed. The severity of the psoriasis can vary widely, approximately 80% of psoriasis patients exhibit mild symptoms with skin plaques covering less than 10% of the body surface area. Whereas, some patients have moderate to severe disease symptoms with skin plaques covering greater than 10% of body surface area (Laws & Young, 2010)

The pathogenesis of psoriasis is linked to various cellular mechanism and the role of T cells, antigen presenting cells (APCs), keratinocytes, Langerhans cells, macrophages, natural killer cells, an array of Th1 type cytokines, as well as certain growth factors like vascular endothelial growth factor (VEGF), keratinocytes growth factor (KGF).

There is no known cure for psoriasis and the current treatment approach advocates that the type of therapy prescribed should be based on appropriateness to disease severity. Amid several treatment options for psoriasis, either systemic or topical agents remain a key component of psoriasis disease management in such patients. The topical agents can be used to treat mild to moderate and moderate to severe psoriasis disease and can potentially reduce the amount of phototherapy or systemic agents required to achieve the effective treatment.

Topical therapies, currently used to treat mild to moderate psoriasis, contain different agents including, topical retinoids (Tazarotene), topical vitamin D analogues (calcitriol, tacalcitol, and calcipotriol), topical corticosteroids, calcineurin inhibitors (pimecrolimus and tacrolimus), emollients, (keratolytics (salicylic acid, urea), dithranol and tars (Murphy and Reich, 2011).

Topical steroids contain ingredients that mimic naturally occurring corticosteroid hormones produced by our adrenal glands. They are designed to be applied externally to the scalp or skin, depending on the condition being treated. Corticosteroids remain a first-line treatment in the management of all grades of psoriasis, both as monotherapy or as a complement to systemic therapy. They are available in a wide range of preparations including gel, cream, ointment, foam, lotion, oil and spray, and a new and innovative vehicle. They are very effective at reducing inflammation (redness and swelling), suppressing the immune system, and constricting (narrowing) blood vessels in the skin. They are used in the treatment of bites, irritation, itching, or rash. Topical steroids come in various potencies (strengths), ranging from ultra-high potency (Class 1) to low potency (Class 7).

The effects of topical corticosteroids are related to four main mechanisms of action: anti-inflammatory, immunosuppressive, antiproliferative, and vasoconstrictive effects. Together, the effects of these medications make them instrumental in treating a wide variety of disorders (Williams, 2005). It has been recommended that super potent (Class 1) topical corticosteroids such as Clobetasol and Halobetasol propionate not to be used for a duration greater than 14 days with the total dose not exceeding 50 grams per week for Halobetasol and 60 grams per week for clobetasol (Habif, 2010). Use under occlusion of these compounds is not recommended. Usage of betamethasone dipropionate should not exceed 45 gms per week (Habif, 2010). There are no guidelines for other classes, but the general recommendation is to switch over to a corticosteroid of lower potency or non-steroidal preparations for maintenance therapy after 2 weeks.

However, many current topical treatments of psoriasis (e.g., steroids) have significant issues including skin damage, such as skin thinning, changes in pigmentation, easy bruising, stretch marks, redness and dilated surface blood vessels. Furthermore, steroids can be absorbed through the skin and affect internal organs when applied to widespread areas of skin, used over long periods of time, or used with excessive occlusion.

In addition, systemic medications (methotrexate, cyclosporine, retinoids, 6-thioguanine, mycophenolate mofetil, troglitazone and new biologic agents, such as adalimumab, alefacept, efalizumab, etanercept, infliximab), and phototherapy or combinations of those are also used to treat psoriasis (Rahman et al., 2012). However, biologics are of limited effectiveness in many patients and, generally, can be used only for a limited duration. Due to immunosuppressive, antiproliferative and anti-inflammatory properties, topical corticosteroids remain as a mainstay topical treatment for various dermatosis conditions including psoriasis (Castela et al., 2012). However, their adverse effects (e. g. atrophy, stria, hypertrichosis, steroid acne, perioral dermatitis, erythema, and telangiectasia) and systemic adverse effects limit their long term and extensive use.

Thus, there remains a need for safe and efficacious treatment of inflammatory skin disorders like psoriasis and treatment regimen developed with low to nil side effects is more likely to produce a satisfactory clinical outcome (Reich and Bewley, 2011).

Anti-inflammatory treatment can be achieved by targeting certain receptors such asIL-R, TLR, Cannabinoid Receptors (CB1R/CB2R) and Peroxisome Proliferator-activated Receptor (PPARs).

Cannabinoids and their derivatives target a broad range of pharmacological mechanisms including anti-inflammatory activity, inhibit keratinocyte proliferation and modulation of the endocannabinoid system. Many of these cannabinoids do not have the psychotropic effects typically associated with cannabis. Therefore, the pharmaceutical products containing specific cannabinoid molecules hold great potential in the field of psoriasis management.

The mechanism of action of cannabinoids as an anti-psoriatic agent is not clear. However, cannabinoids are found to interact with a variety of receptors resulting in modulatory effects. The receptors include, but not limited to, cannabinoid receptor (CB₁ and CB₂), transient receptor potential cation channel (TRPV1, TRPV2, TRPA1, TRPM8), PPAR, α₃ glycine receptors, G-protein coupled receptor GPR-55, serotonin receptors, adenosine Ai receptors, a2 adrenoceptors, and endocannabinoid (anandamide) reuptake.

Agonists of PPARs have been reported as a promising drug target for liver diseases and inflammatory skin conditions (e.g. psoriasis, atopic dermatitis, and skin malignancies). PPARs are a subfamily of ligand-inducible nuclear hormone transcription factors comprising of the following three subtypes: PPARα, PPARβ (also known as PPARδ), and PPARγ. While they are best known as transcriptional regulators of lipid and glucose metabolism, evidence has also accumulated for their importance in skin homeostasis. it has been reported that in inflammatory skin disorders, the expression of both PPARα and PPARγ is decreased. This observation suggests the possibility that PPARα and PPARγ activators, or compounds that positively regulate PPAR gene expression, may represent novel treatment for the topical or systemic treatment of common inflammatory skin diseases and disorders.

### SUMMARY OF THE INVENTION

This disclosure relates to novel molecules, compounds and pharmaceutically acceptable salts thereof, methods, and formulations in a delivery system for the prevention and treatment of inflammation and/or PPAR mediated diseases or conditions. The PPAR mediated diseases or conditions may comprise skin disorders, peripheral diseases, steatohepatitis, type 2 diabetes, Alzheimer's disease, Parkinson's disease, multiple sclerosis, diabetes, metabolic syndromes, hyperlipemia, high-blood pressure, vascular disorders, dermatitis, psoriasis, inflammation, hepatitis, fatty liver, liver fibrosis, non-alcoholic steatohepatitis (NASH), liver cancer, cirrhosis, primary biliary cirrhosis, viral hepatitis, hepatic fibrosis, insulin resistance, impaired glucose tolerance, hyperinsulinemia, hypertriglyceridemia, non-alcoholic fatty liver disease, type 1 diabetes, hypercholesterolemia, nephropathy, pancreatitis, nephritis, stroke, arteriosclerosis, atherosclerosis, coronary heart disease, eczema, impaired wound healing, asthma, Crohn's disease, inflammatory bowel syndrome, ophthalmic inflammation, rheumatoid arthritis, and/or neurodegenerative disorders.

In one aspect the PPAR mediated diseases or conditions may include skin disorders and their symptoms. More specifically, the present disclosure relates to novel molecules, compounds or formulations in a delivery system, that may be combined with cannabinoids to treat PPAR mediated diseases or conditions such for example inflammatory skin disorders. The skin disorders may be for example psoriasis and related autoimmune inflammatory skin disorders.

In one aspect, there is provided a compound of formula I

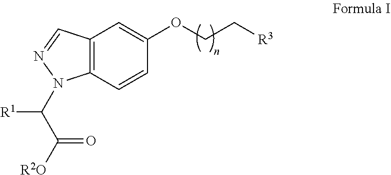

or a pharmaceutically acceptable salt, stereoisomer, or solvate thereof,
wherein
n is selected from 1 or 2;
R¹ is selected from H, OC₂H₅, or O-alkyl;
R² is selected from H, CH₃, or alkyl chain;
R³ is selected from cycloalkyl, cycloalkylene, phenyl, substituted phenyl, phenoxyl, substituted phenoxyl, heterocyclyl, or substituted heterocyclyl, amine, or substituted amine.

In some embodiments: n is 1 or 2; R¹ is H; R² is H or CH₃; and R³ is selected from:

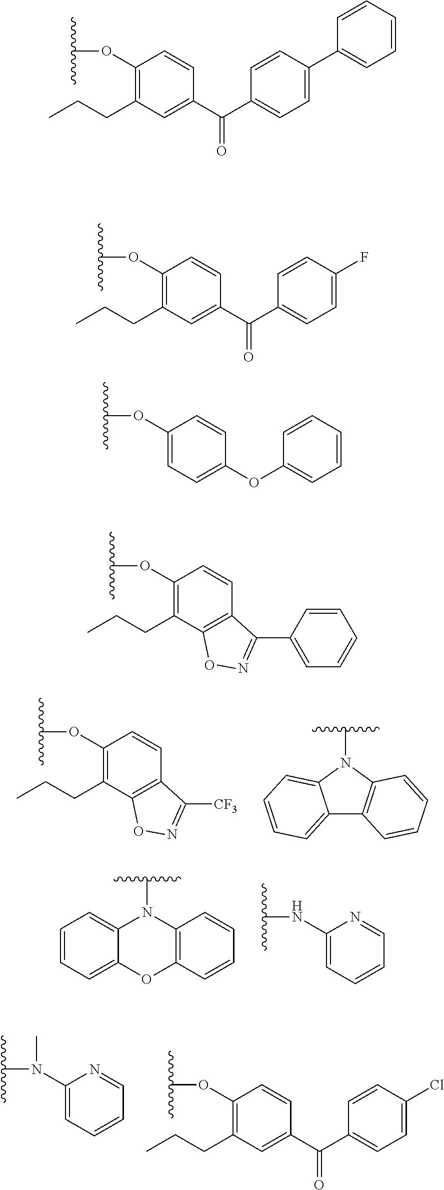

In some embodiments, n is 1 or 2; R¹ is OC₂H₅; R² is H or CH₃; and R³ is selected from:

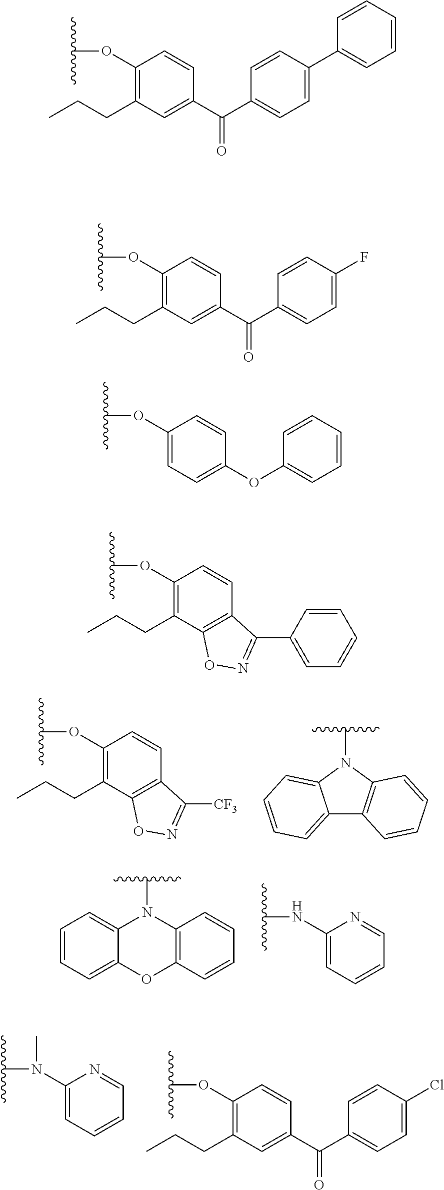

In some embodiments, n is 1. In alternative embodiments, n is 2.

The compound may be selected from the group consisting of:

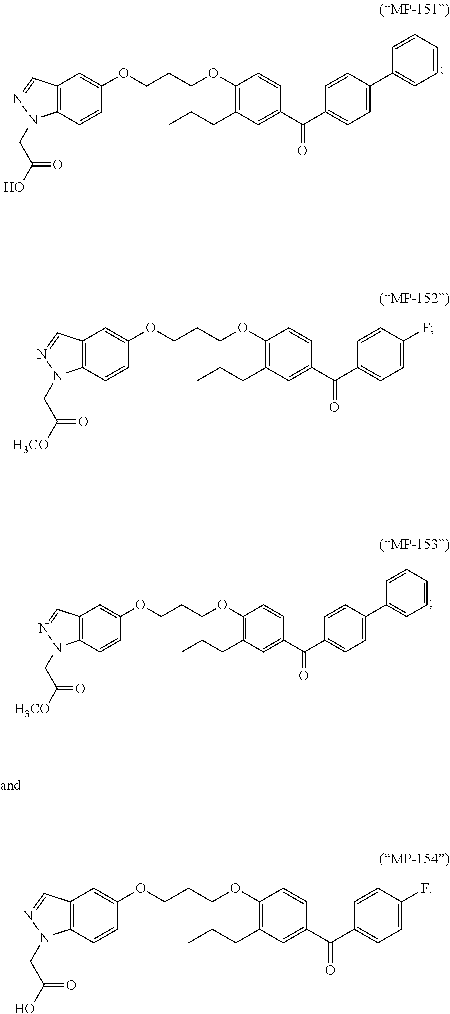 [SMILES: CCCc1cc(C(=O)c2ccc(-c3ccccc3)cc2)ccc1OCCCOc1ccc2c(cnn2CC(=O)O)c1 ; CCCc1cc(C(=O)c2ccc(F)cc2)ccc1OCCCOc1ccc2c(cnn2CC(=O)OC)c1 ; CCCc1cc(C(=O)c2ccc(-c3ccccc3)cc2)ccc1OCCCOc1ccc2c(cnn2CC(=O)OC)c1 ; CCCc1cc(C(=O)c2ccc(F)cc2)ccc1OCCCOc1ccc2c(cnn2CC(=O)O)c1]

The pharmaceutically acceptable salt may be a hydrochloric acid, sulfuric acid, sulfonic acid, nitric acid, phosphoric acid, acetic acid, oxalic acid, propionic acid, succinic acid, maleic acid, tartaric acid or citric acid salt.

In another aspect, there is provided a pharmaceutical composition comprising one or more compounds of formula I as described herein and one or more pharmaceutically acceptable excipients. In some embodiments, the compound is in the form of a prodrug, salt, solvate or ester thereof

In some embodiments, the pharmaceutical composition further comprises one or more cannabinoid. The cannabinoid may comprise cannabidiol (CBD) or Abnormal Cannabidiol (Abn-CBD) or their prodrug or pharmaceutically acceptable salts thereof.

The one or more pharmaceutically acceptable excipients may be selected from protectives, adsorbents, demulcents, emollients, preservatives, antioxidants, moisturizers, buffering agents, solubilizing agents, skin-penetration agents, surfactants, physiologically acceptable surface-active agents, carriers, penetrating agents, diluents, smoothing agents, suspension agents, film forming substances, coating assistants, and combinations thereof.

The pharmaceutical composition may be formulated for topical administration. The pharmaceutical composition may comprise at least 0.0001% by weight of the one or more compound of formula I. The pharmaceutical composition may comprise between 0.0001% and 15% by weight of the one or more compound of formula I.

In another aspect, there is provided the compound or pharmaceutical composition described herein for use in treating a skin disorder.

In another aspect, there is provided a method for treating a PPAR mediated diseases or conditions such for example a skin disorder in a subject, the method comprising administering the compound or the pharmaceutical composition described herein to the subject.

The skin disorder may be an inflammatory and/or an autoimmune skin disorder. The skin disorder may comprise generalized topical skin wounds and wherein the treatment or use may comprise wound healing of the topical skin wounds. The inflammatory and/or an autoimmune skin disorder may be psoriasis, psoriatic arthritis, atopic dermatitis, inflammation, eczema or dermatitis. The psoriasis may be selected from the group consisting of plaque, guttate, inverse, pustular, and erythrodermic psoriasis.

In some embodiments, the method of treating or use may comprise a protracted or an extended treatment period. For example, the treatment period may be at least 1 day, at least 2 days, at least 3 days, at least 4 days, at least 5 days, at least 6 days, at least 1 week, at least 2 weeks, at least 3 weeks, at least 1 month, at least 2 months, at least 3 months, at least 4 months, at least 5 months, at least 6 months or at least 12 months, or any amount therebetween. For example, the treatment period may be between 1-7 days, 1-14 days, 1-21 days, 1-28 days or 1 month to 1 year.

In some embodiments, the method of treating or use may comprise a protracted treatment period. The protracted treatment period may comprise between about 1 to about 60 days or any number of days therebetween. For example, the protracted treatment period may be between about 1 day to about 1 month.

In some embodiments, the compound or pharmaceutical composition may be administered to a dermal surface, transdermal surface, mucosal surface, transmucosal surface or a combination thereof. The transmucosal surface may be sublingual, buccal, nasal, ocular, vaginal, and/or rectal mucosae. In some embodiments, the compound or pharmaceutical composition is topically administered to the skin or mucosa.

In some embodiments, the compound or the pharmaceutical composition is formulated for delivery as an aerosol, liquid, gel, or tablet/solid drug carrier, capsules, creams, ointments, lotions, hydrogels, pastes, dusting powders, salve, sponges, implants, foams, emulsions, sprays, viscous liquids, shampoos, semisolids, patches, occlusive dressings such as a medicated band-aid or gauze, films, transdermal therapeutic systems or discs which releases the active ingredient at a predetermined rate over a defined period of time to a defined site of application, liposomes, iontophoresis, electroporation, electrospinning, phonophoresis, sonophoresis, needle-free or microneedle injection.

In another aspect, there is provided a combination of a PPAR agonist and a cannabinoid, for use in treating a skin disorder. The PPAR agonist may be the compound of formula I described herein. The cannabinoid may be, but not limited to, CBD, Abn-CBD or their prodrug or pharmaceutically acceptable salts thereof. The skin disorder may be an inflammatory and/or autoimmune skin disorder. The inflammatory and/or autoimmune skin disorder may be psoriasis, psoriatic arthritis, atopic dermatitis, inflammation, eczema or dermatitis. The combination may be formulated as a topical composition.

The pharmaceutical composition may be formulated for topical administration in combination with a compound of Formula I. The pharmaceutical composition may comprise at least 0.0001% by weight of the one or more cannabinoids. The pharmaceutical composition may comprise between 0.0001% and 15% by weight of the one or more cannabinoids.

### BRIEF DESCRIPTION OF THE DRAWINGS

The present invention will be further illustrated by the following examples. The following are examples for activity demonstration purposes and should not limit the scope of claims and applications of the invention.

These and other features of the invention will become more apparent from the following description in which reference is made to the appended drawings wherein:

FIG. 1 Cell-based In vitro human PPARα activation of novel PPAR agonists

FIG. 2 Cell-based in vitro human PPARδ/β activation of novel PPAR agonists

FIG. 3 Cell-based in vitro human PPARγ activation of novel PPAR agonists

FIG. 4 In vitro cytotoxicity of novel PPAR agonists.

FIGS. 5A-5B Anti-inflammatory effect of novel PPAR agonists on cytokine production in IMQ-stimulated HaCaT cell model. FIG. 5A The IL-6 levels after drug treatments for 24 h. FIG. 5B The IL-8 levels after drug treatments for 24 h. C-DMSO: DMSO control. C-IMQ: IMQ control.

FIGS. 6A-6B Anti-inflammatory effect of novel PPAR agonists on cytokine production in TNFα/IL-17A -stimulated HaCaT cell model. FIG. 6A shows IL-6 levels after drug treatments for 24 h. FIG. 6B shows IL-8 levels after drug treatments for 24 h. C-DMSO: DMSO control. C-TNFa+IL-17A: TNFa+IL-17A control.

FIGS. 7A-7B Anti-inflammatory effect of novel PPAR agonists on cytokine production in LPS-stimulated HaCaT cell model. FIG. 7A shows IL-6 levels after drug treatments for 24 h. FIG. 7B shows IL-8 levels after drug treatments for 24 h. C-DMSO: DMSO control. C-LPS: LPS control.

FIGS. 8A-8B Inhibition of inflammatory cytokine production in HaCaT cells by combined treatments of CBD and PPAR agonist (MP-151). FIG. 8A shows IL-6 levels after drug treatments for 24 h by CBD, MP-151, and combination of CBD and MP-151. FIG. 8B shows IL-8 levels after drug treatments for 24 h by CBD, MP-151, and combination of CBD and MP-151.

FIG. 9 Inhibition of inflammatory cytokine IL-8 production by CBD, MP-152, and combination of CBD and PPAR agonist (MP-152).

FIG. 10 Inhibition of inflammatory cytokine IL-6 production by Abnormal cannabidiol (Abn-CBD), MP-152, and combination of Abn-CBD and MP-152.

FIG. 11 Time-course pattern of IMQ-induced psoriasis as represented by the Psoriasis Area and Severity Index (PASO score in BALB/c mice treated with CBD and the PPAR agonist compounds (MP-151 and MP-152).

FIG. 12 Comparison of PASI scores in IMQ-induced psoriasis model in BALB/c mice on day 11 and 17.

### DETAILED DESCRIPTION

This disclosure relates to design, synthesis, and formulation of novel compounds, in particular novel PPAR agonist compounds. The disclosure further relates to pharmaceutical formulations comprising PPAR agonists and cannabinoids. The disclosure further relates to uses and methods of the novel compounds and pharmaceutical compositions for the prevention and treatment of inflammatory diseases, such as skin disorders.

The compounds according to the present disclosure may have intrinsic PPAR agonist properties. They may be therefore of particular interest in the treatment of PPAR mediated disease or condition such for example metabolic and/or inflammatory diseases, such as skin disorders, peripheral diseases and central diseases associated with the metabolic syndrome, such as diverse forms of steatohepatitis, type 2 diabetes, diverse neurodegenerative disorders such as Alzheimer's disease, Parkinson's disease, multiple sclerosis, diabetes, metabolic syndromes, hyperlipemia, high-blood pressure, vascular disorders, dermatitis, psoriasis, inflammation, hepatitis, fatty liver, liver fibrosis, NASH (non-alcoholic steatohepatitis) and/or obesity. Therefore, the effects of the compounds of the present disclosure on the above diseases are expected beyond those of the existing therapeutic agents.

Furthermore, it provides a method of treating a PPAR mediated disease or condition. For example the PPAR mediated disease or condition may be a skin disorders, peripheral diseases and central diseases associated with the metabolic syndrome, such as diverse forms of steatohepatitis, type 2 diabetes, diverse neurodegenerative disorders such as Alzheimer's disease, Parkinson's disease, multiple sclerosis, a disease selected from the group consisting of diabetes, metabolic syndromes, hyperlipemia, high-blood pressure, vascular disorders, inflammation, skin disorders, hepatitis, fatty liver, liver fibrosis, non-alcoholic steatohepatitis, obesity, liver cancer, cirrhosis, primary biliary cirrhosis, viral hepatitis, hepatic fibrosis, insulin resistance, impaired glucose tolerance, hyperinsulinemia, hypertriglyceridemia, non-alcoholic fatty liver disease, type 1 diabetes, hypercholesterolemia, nephropathy, pancreatitis, nephritis, stroke, arteriosclerosis, atherosclerosis, coronary heart disease, eczema, impaired wound healing, asthma, Crohn's disease, inflammatory bowel syndrome, ophthalmic inflammation, rheumatoid arthritis, and/or neurodegenerative disorders. The method comprising administering an effective amount of a peroxisome proliferator-activated receptor activity regulator to a subject in need thereof.

The description is directed to a pharmaceutical composition or formulation for treating a skin disorder. The formulation may comprise an effective amount of one or more than one PPAR agonist. The formulation may further comprise one or more than one cannabinoid, for example the cannabinoid may be a CB1/CB2 receptor agonist.

The present disclosure further provides methods, compounds, and topical skin formulations for treatment of inflammatory skin disorders and their symptoms. The methods, compounds, and formulations of the disclosure are particularly effective for treatment of psoriasis, but can be used to treat other inflammatory skin diseases including but not limited to disorders of hair follicles and sebaceous glands, such as acne, rosacea and rhinophyma; dermatitis, such as contact dermatitis, atopic dermatitis, seborrheic dermatitis, perioral dermatitis; and inflammatory reactions, such as drug eruptions, erythema multiforme, granuloma annulare, and erythema nodosum.

The skin disorder may comprise inflammatory skin conditions, psoriasis, psoriatic arthritis or atopic dermatitis. The psoriasis may be selected from the group consisting of plaque, guttate, inverse, pustular, and erythrodermic.

The skin disorder may be for example psoriasis, psoriatic arthritis or atopic dermatitis.

Furthermore, generalized topical skin wounds and wounds may be associated with the skin disorder, and the treatment comprises wound healing of the topical skin wounds and wounds associated with skin disorder. The skin disorder may comprise inflammation, psoriasis, eczema and dermatitis.

To treat or prevent inflammatory skin disorders, according to the methods of the disclosure, the compounds of this disclosure may be topically applied. In some embodiments, the compounds of the disclosure may be delivered in a topical formulation. For example, the compounds of the present disclosure may be formulated for topical delivery. For example, the compounds may be formulated as aqueous or non-aqueous solutions or suspensions, emulsions, creams, lotions, gels, or ointments.

Furthermore, it is provided a pharmaceutical preparation for treating psoriasis, psoriatic arthritis, atopic dermatitis, and wounds comprising the formulation as described above and physiologically acceptable surface-active agents, carriers, penetrating agents, diluents, excipients, smoothing agents, suspension agents, film forming substances, coating assistants, or a combination thereof.

The formulation may be administered to a dermal surface, transdermal surface, mucosal surface, transmucosal surface or a combination thereof. The transmucosal surface may be sublingual, buccal, nasal, ocular, vaginal, and/or rectal mucosae. The formulation may be topically administered to the skin or mucosa. The formulation may be delivered in an appropriate aerosol, liquid, gel, or tablet/solid drug carrier, capsules, creams, ointments, lotions, hydrogels, pastes, dusting powders, salve, sponges, implants, foams, emulsions, sprays, viscous liquids, shampoos, semisolids, patches, occlusive dressings such as a medicated band-aid or gauze, films, transdermal therapeutic systems or discs which releases the active ingredient at a predetermined rate over a defined period of time to a defined site of application, liposomes, iontophoresis, electroporation, phonophoresis, sonophoresis, needle-free or microneedle injection.

### PPAR Agonists

Immunosuppressive effects of agonists of PPARs have been reported as a promising drug target for inflammatory skin diseases (e.g. psoriasis, atopic dermatitis, eczema, acne vulgaris, and other dermatitides), liver diseases (e.g. NASH, hepatitis, fatty liver, cirrhosis), neurodegenerative diseases (e.g. multiple sclerosis, Alzheimer's disease, Parkinson's disease), cardiovascular diseases (e.g. atherosclerosis, venous and arterial occlusive diseases, restenosis after invasive procedures, cardiomyopathy, myocardial fibrosis, congestive heart failure), pulmonary disorders (asthma, chronic obstructive pulmonary disease), angiogenesis and neovascularization in neoplastic and other diseases.

The term “Peroxisome proliferator-activated receptors agonist” or “PPAR agonist” refers to a substance, composition, compound or molecule that acts on one or more than one peroxisome proliferator-activated receptors, such for example on PPARα (alpha), PPARγ (gamma) or PPARδ (delta). Peroxisome proliferator-activated receptors (PPARs) are members of the nuclear receptor superfamily of ligand-activated transcription factors. PPARs are a family of 3 ligand-activated transcription factors: PPAR alpha (NR1C1), PPAR delta (also known as PPAR beta, NUC1, NR1C2), and PPAR gamma (NR1C3). PPAR alpha, delta, and gamma are encoded by different genes but show substantial amino acid similarity, especially within the DNA and ligand binding domains.

Accordingly, a PPAR agonists may be a PPAR-alpha agonist, a PPAR-gamma agonist, a PPAR-delta agonist, a dual PPAR agonist binding to any two of the three PPAR isoforms, or a pan-PPAR agonist binding to all three PPAR receptors.

Non-limiting examples of PPAR agonists may include thiazolidinediones or thiazolidinedione derivatives such for example rosiglitazone, pioglitazone or ciglitazone.

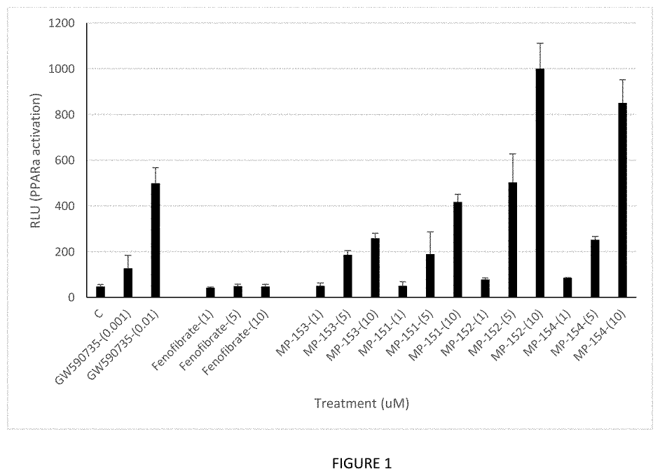

The compounds described herein of formula I have been shown to have PPAR agonist activity (see FIGS. 1-3).

The PPAR agonist may be a compound of formula I:

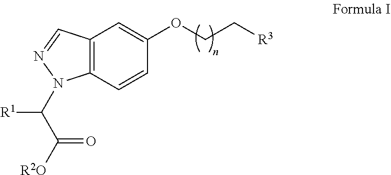

wherein
n is selected from 1 or 2;
R¹ is selected from H or OC₂H₅;
R² is selected from H or CH₃;
R³ is selected from cycloalkyl, cycloalkylene, phenyl, substituted phenyl, phenoxyl, substituted phenoxyl, heterocyclyl, or substituted heterocyclyl, amine, or substituted amine.

The PPAR agonist compound may be defined as follows:

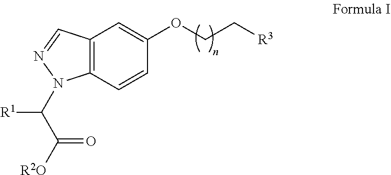

wherein
n is selected from 1 or 2;
R¹ is selected from H or OC₂H₅;
R² is selected from H or CH₃;
R³ is selected from cycloalkyl, cycloalkylene, phenyl, substituted phenyl, phenoxyl, substituted phenoxyl, heterocyclyl, or substituted heterocyclyl, amine, or substituted amine.

In some embodiments, n is 1. In alterantive embodiments, n is 2. In some embodiments, R¹ is H. In some embodiments, R¹ is OC₂H₅. In some embodiments, R² is H. In some embodiments, R² is CH₃.

In some embodiments, R³ is phenyl. In some embodiments, R³ is substituted phenyl. In some embodiments, R³ is phenoxyl. In some embodiments, R³ is substituted phenoxyl. In some embodiments, R³ is heterocyclyl. In some embodiments, R³ is substituted heterocycicyl. In some embodiments, R³ is amine. In some embodiments, R³ is substituted amine.

In some embodiments, R³ may be one of the following:

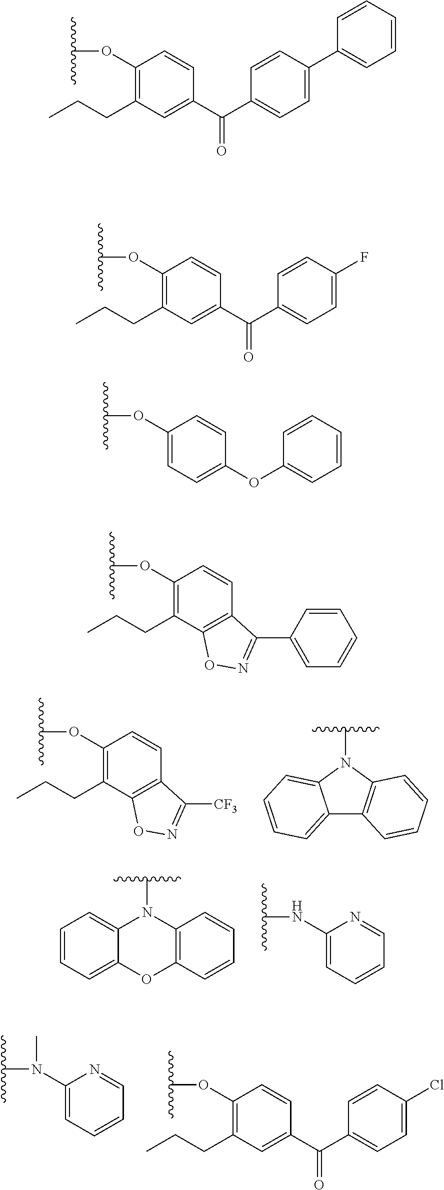

In some embodiments, R³ is

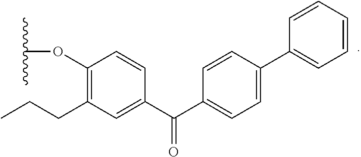 [SMILES: *Oc1ccc(C(=O)c2ccc(-c3ccccc3)cc2)cc1CCC]

In some embodiments, R³ is

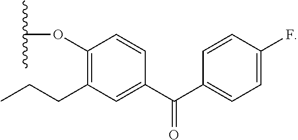 [SMILES: *Oc1ccc(C(=O)c2ccc(F)cc2)cc1CCC]

In some embodiments, the compound may be defined as follows:

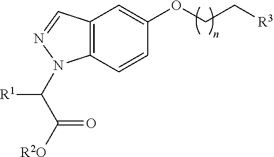

wherein:
n may be 1;
R¹ is selected from H or OC₂H₅;
R² is selected from H or CH₃;
R³ may be selected from:

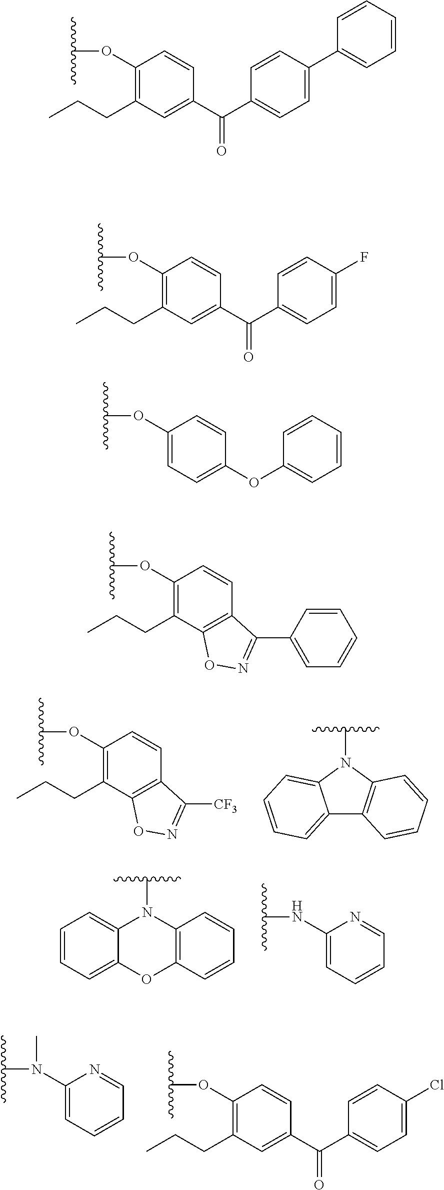

In some embodiments, the compound may be defined as follows:

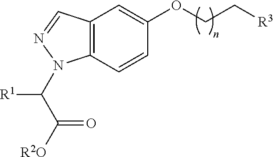

wherein
n may be 2;
R¹ is selected from H or OC₂H₅;
R² is selected from H or CH₃;
R³ may be selected from:

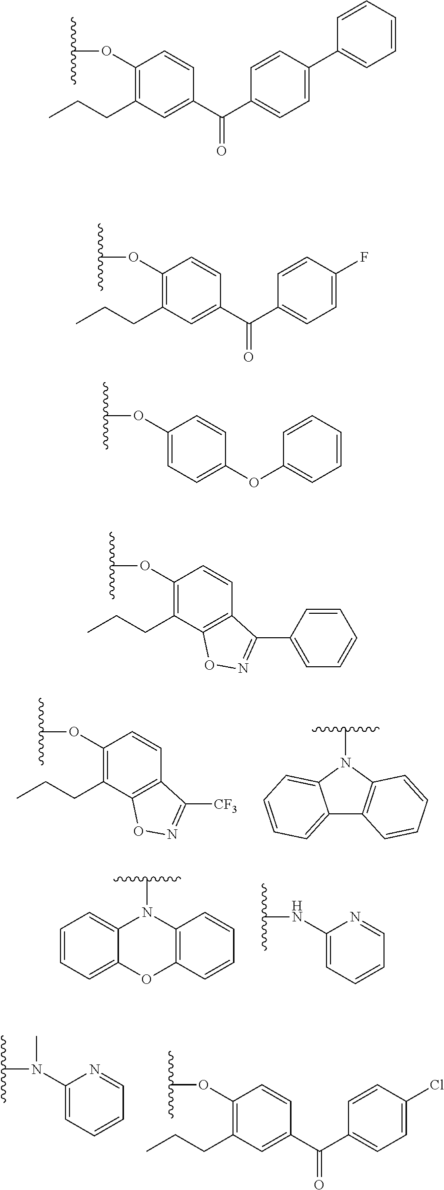

In another embodiments, the compound may be defined as follows:

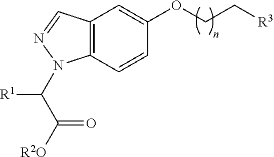

wherein
n may be 1 or 2;
R¹ is H;
R² is selected from H or CH₃;
R³ may be selected from:

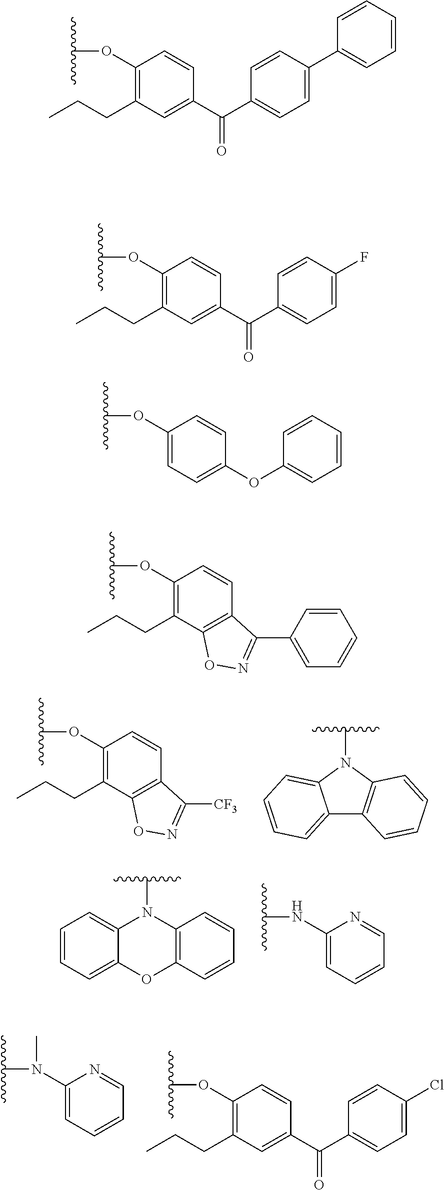

In some embodiments, the compound may be defined as follows:

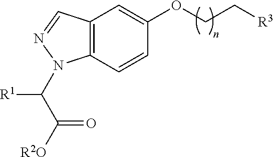

wherein
n may be 1 or 2;
R¹ is OC₂H₅;
R² is selected from H or CH₃;
R³ may be selected from:

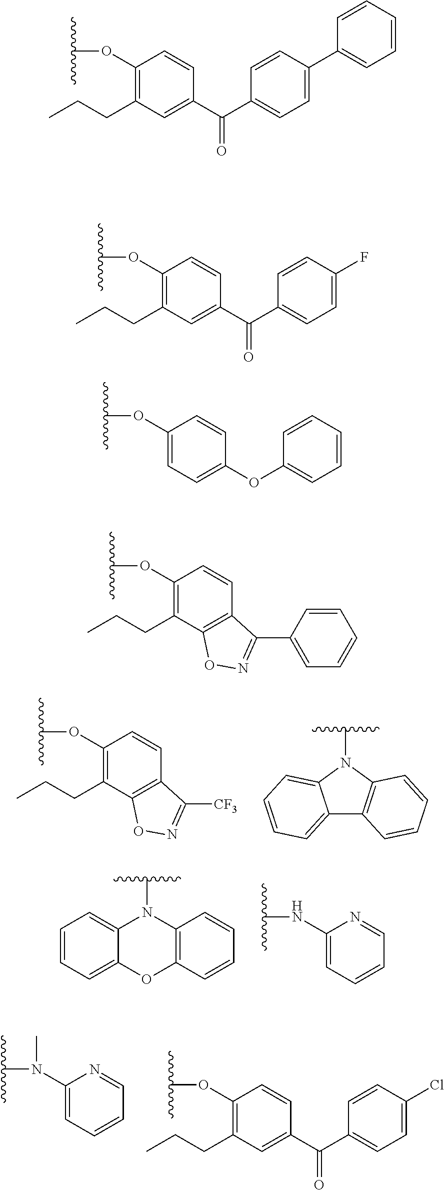

The “alkyl” may be a C₁₋₆ alkyl. The alkyl may be methyl, ethyl, propyl, isopropyl, butyl, isobutyl, sec-butyl, tert-butyl, pentyl, isopentyl, neopentyl, tert-pentyl, 1-methylbutyl, 2-methylbutyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1-ethyl-2-methylpropyl, 1,1,2-trimethylpropyl and the like. The alkyl group having a carbon number of 1-6 is preferably methyl.

The term “homocyclyl” refers to a cyclic compound where all ring members are the same chemical element. The homocyclyl may be a carbocyclyl, i.e. where all atoms are carbon.

The term “phenyl” refers to a cyclic group of atoms with the formula C₆H₅.

The term “phenoxyl” refers to the substituent —O-phenyl (or —O—C₆H₅).

The term “heterocyclyl” refers to a cyclic compound where at least one ring member is not a carbon atom. For example, the heterocyclyl may be an N-containing, S-containing or O-containing heterocycle.

The term “amine” refers to the group —NR₂, where each R may be selected from H or a substitution.

The substitutions on the R³ groups may be any suitable substituent. For example, the substitutions on R³ may comprise one or more selected from the group consisting of alkyl such as a C₁₋₆ alkyl, haloalkyl such as C₁₋₆ haloalkyl, cyclyl, homocyclyl, heterocyclyl, carbonyl, ether, amine, amide, and fluorocarbon.

For example, R³ may be selected from:

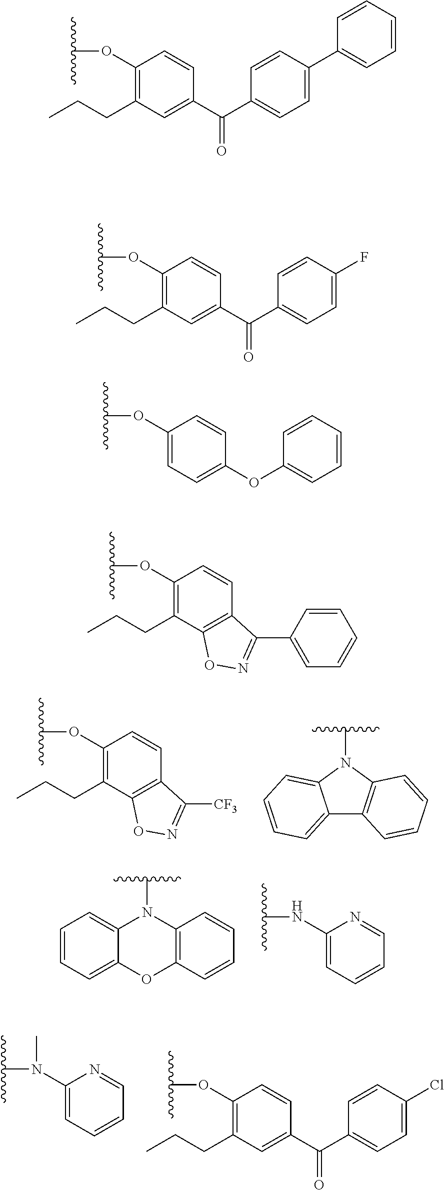

In some embodiments, n is 1. In alternative embodiments, n is 2. In some embodiments, R¹ is H. In alternative embodiments, R¹ is OC₂H₅. In some embodiments, R² is H. In alternative embodiments, R² is CH₃.

In some embodiments, the compositions or formulations described herein may comprise at least 0.0001%, at least 0.001%, at least 0.01%, at least 0.1% or at least 0.5% by weight of one or more PPAR agonist compounds, e.g. the compound of formula I described herein. The compositions or formulations may comprise between 0.0001% and 15%, between 0.001% and 15%, between 0.01% and 15%, between 0.1% and 15%, between 0.5% and 15% or between 0.5% and 10% by weight of one or more PPAR agonist compounds. The compositions or formulations may comprise about 1% by weight of one or more PPAR agonist compounds. The compositions or formulations may comprise about 5% by weight of one or more PPAR agonist compounds. The composition may further contain one or more cannabinoids. PPAR agonist to cannabinoid ratio may be 0.5:1-1:100 or any ratio therebetween. For example, the PPAR agonist to Cannabinoid ratio may be 0.5:1, 1:1, 1:2, 1:5, 1:10, 1: 50, 1:100, or any ratio therebetween. Furthermore, the cannabinoid to PPAR ratio may be 0.5:1 to 1:100 or any ratio therebetween. For example, the cannabinoid to PPAR ration may be 0.5:1, 1:1, 1:2, 1:5, 1:10, 1: 50, 1:100, or any ratio therebetween.

The formulation may comprise an effective amount of PPAR agonist between about 100 ng (0.0001 mg) to about 1000 mg, such as between 0.001 mg and about 500 mg.

The present disclosure also relates to processes for the preparation of the above compounds as described above, their derivatives, their analogs, their tautomeric forms, their stereoisomers, their polymorphs, their pharmaceutically acceptable salts or pharmaceutically acceptable solvates.

### Cannabinoids

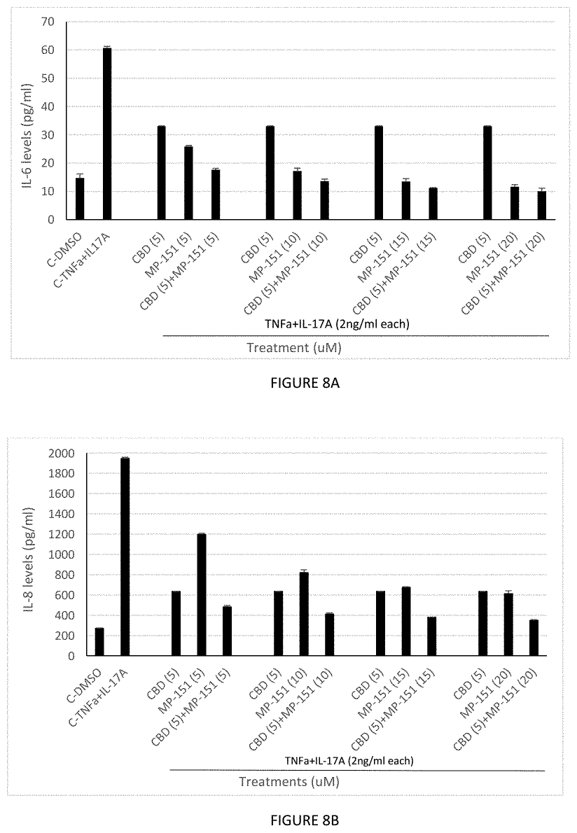

The compositions, formulations and combinations described herein may comprise one or more cannabinoid, its prodrugs, or pharmaceutically acceptable salts, thereof. It has been found that the combination of a PPAR agonist compound and a cannabinoid significantly reduced skin inflammation compared to the PPAR agonist or cannabinoid when administered alone (see FIGS. 8-10).

The one or more than one cannabinoid may comprise CBD, Abn-CBD, tetrahydrocannabinol (THC), Cannabigerol (CBG), endocannabinoids (e.g. N-arachidonoylethanolamine (AEA), 2-Arachidonoylglycerol (2-AG)) or a combination thereof. In some embodiments, the cannabinoid comprises CBD. In some embodiments, the cannabinoid comprises Abn-CBD.

The compositions, formulations and combinations may comprise an effective amount of CBD or Abn-CBD between 0.001 mg and 1000 mg.

By the term “cannabinoid” it is meant, a substance, composition, compound or molecule that act on one or more than one cannabinoid receptor, such for example cannabinoid receptor type 1 (CB₁) or cannabinoid receptor type 2 (CB₂) or may otherwise interact or modulate the endocannabinoid system or other cannabinoid-binding proteins such as for example transient receptor potential cation channels (e.g., TRPV1, TRPV2, TRPA1, TRPM8), GPR (e.g., GPR55, GPR18, GPR119) receptors, serotonin receptors (e.g., 5-HT1A), endocannabinoid transporter and reuptake proteins, α3 glycine receptors, adenosine A1 receptors or α2 adrenoceptors. Cannabinoid receptors may be activated by four major groups of ligands, endocannabinoids (produced by the mammalian body), phytocannabinoid or plant cannabinoids (produced by plants for example the cannabis plant), synthetic cannabinoids (produced artificially), and cannabimimetic compounds. In one example, a cannabinoid may be a substance, composition, compound or molecule that acts on CB₁ and/or CB₂ receptors.

In some embodiments, the cannabinoid may be a phytocannabinoid. For example, the cannabinoid may be extracted and/or purified from cannabis plants.

The term “cannabis plant(s)” encompasses wild type Cannabis sativa and variants thereof, including cannabis chemovars (varieties characterized by virtue of chemical composition) which naturally contain different amounts of individual cannabinoids, also Cannabis sativa subspecies indica including the variants var. indica and var. kafiristanica, Cannabis indica and also plants which are the result of genetic crosses, self-crosses or hybrids thereof. Furthermore, cannabis plants include for example the species Cannabis sativa, Cannabis indica or Cannabis ruderalis. The term “cannabis plant material” is to be interpreted accordingly as encompassing plant material derived from one or more cannabis plants. For the avoidance of doubt, it is hereby stated that “cannabis plant material” includes herbal cannabis and dried cannabis biomass.

For example, cannabis plant material derived from cannabis plants having a relatively high content of CBD (as CBDA and/or CBD) may be used. For example, cannabis varieties (chemovars) having a CBDA/CBD content of >90% of the total cannabinoid content may be used. In particular, phytocannabinoid may be extracted or purified for example from high CBD strains such for example Charlotte's Web or Avidekal.

Non-limiting examples of phytocannabinoids include CBD or CBD derivatives, Cannabigerol (CBG), Cannabichromene (CBC), Cannabicyclol (CBL), Cannabivarin (also known as cannabivarol or CBV), Tetrahydrocannabivarin (THCV, THV), Cannabidivarin (CBDV), Cannabichromevarin (CBCV), Cannabigerovarin (CBGV), Cannabigerol Monomethyl Ether (CBGM). In one non-limiting example, the cannabinoid may be a CBD extracted and purified from one or more high CBD strain such for example Charlotte's Web or Avidekal.

In another example, the cannabinoid may be a synthetic cannabinoid (also known as synthetic cannabis (synthetic marijuana), or synthetic cannabinoid receptor agonists), for example Cannabicyclohexanol (CP 47,497), JWH-018, JWH-073, or HU-210, Epigallocatechin, Epicatechin, Kavain, Yangonin, N-Arachidonoyl dopamine, CBD, Abn-CBD, Cannabinol (CBN), HU-210, 11-Hydroxy-49-tetrahydrocannabinol (11-OH-THC), dronabinol or Levonantradol (CP 50,556-1).

In some embodiments, the cannabinoid may be an endocannabinoid. For example, the endocannabinoid may be AEA and 2-AG.

The one or more than one cannabinoid may be from group of phyto-cannabinoids, endocannabinoids or synthetic cannabinoids, or their pharmaceutically acceptable salts, thereof. The one or more than phyto, endo or synthetic cannabinoid may be from the group of THC, CBD, CBG, CBC, THCV, CBN, CBDV, Abn-CBD, AEA, 2-AG or their derivatives or a combination thereof or their prodrug forms. The one or more than one phyto or synthetic cannabinoid may be CBD or their derivatives or prodrugs. The one or more than one phyto or synthetic cannabinoid may be Abn-CBD or their derivatives or prodrugs. The effective amount of CBD or Abn-CBD in the formulation may be between 0.001 mg and 1000 mg or any amount there between.

The formulation as described herein, may comprise an effective amount of one or more than one cannabinoid from about 0.001 mg to about 1000 mg, or any amount there between. For example, the effective amount of the one or more than one cannabinoid may be 0.001 mg, 0.005 mg, 0.01mg, 0.05mg, 0.10mg, 0.5 mg, 1 mg, 2 mg, 3 mg, 4 mg, 5 mg, 10 mg, 20 mg, 30 mg, 40 mg, 50, mg, 100 mg, 200 mg, 300 mg, 400 mg, 500 mg, 600 mg, 700 mg, 800 mg, 900 mg, 1000 mg, or any amount therebetween. The one or more than one cannabinoid may for example be CBD or a CBD derivative. In one example, the formulation described herein may therefore comprise an effective amount of CBD or a CBD derivative from about 0.001 mg to about 1000 mg, or any amount there between. For example, the effective amount of CBD or the CBD derivative may be 0.001 mg, 0.01 mg, 0.1 mg, 0.5 mg, 1 mg, 2 mg, 3 mg, 4 mg, 5 mg, 10 mg, 20 mg, 30 mg, 40 mg, 50, mg, 100 mg, 200 mg, 300 mg, 400 mg, 500 mg, 600 mg, 700 mg, 800 mg, 900 mg, 1000 mg, or any amount therebetween. The one or more than one cannabinoid may for example be Abn-CBD or a Abn-CBD derivative. In one example, the formulation described herein may therefore comprise an effective amount of Abn-CBD or a Abn-CBD derivative from about 0.001 mg to about 1000 mg, or any amount there between. For example, the effective amount of Abn-CBD or the Abn-CBD derivative may be 0.001 mg, 0.01 mg, 0.1 mg, 0.5 mg, 1 mg, 2 mg, 3 mg, 4 mg, 5 mg, 10 mg, 20 mg, 30 mg, 40 mg, 50, mg, 100 mg, 200 mg, 300 mg, 400 mg, 500 mg, 600 mg, 700 mg, 800 mg, 900 mg, 1000 mg, or any amount therebetween.

In some embodiments, the compositions or formulations described herein may comprise at least 0.001% by weight of one or more cannabinoids. The compositions or formulations may comprise between 0.001% and 50% or any amount therebetween by weight of one or more cannabinoids. For example the formulations may comprise 0.001%, 0.01%, 0.1%, 0.5%, 1%, 5%, 10%, 15%, 20%, 30%, 40% or 50% by weight of one or more cannabinoids.

PPAR agonist to Cannabinoid ratio may be 0.5:1-1:100 or any ratio therbetween. For example the PPAR agonist to Cannabinoid ratio may be 0.5:1, 1:1, 1:2, 1:5, 1:10, 1: 50, 1:100, or any ratio therebetween. Furthermore the Cannabinoid to PPAR ratio may be 0.5:1 to 1:100 or any ratio therebetween. For example the Cannabinoid to PPAR ration may be 0.5:1, 1:1, 1:2, 1:5, 1:10, 1:50, 1:100, or any ratio therebetween.

### Purification/Extraction of Cannabinoids

Methods of preparing cannabinoids from plant in substantially pure form starting from plant material are known in the art.

For example, WO 02/064109 which is herein incorporated by reference, describes a general method for obtaining whole extracts from cannabis plant material. WO 02/32420 discloses a process for preparing, for example, Δ⁹-THC from plant material. It utilizes CO₂ extraction and ethanol precipitation to obtain “primary extracts” containing Δ⁹-THC and CBD, with reduced amounts of, for example, monoterpenes, sesquiterpenes, hydrocarbons, alkaloids, flavonoids and chlorophylls. The CBD is then converted to Δ⁹-THC by a catalyzing reaction. ODCCP Bulletin on Narcotics (1976, Issue 4) discloses a method of isolating CBD (Repetto et al, 1976), THC and CBN using preparative gas chromatography. ODCCP Bulletin on Narcotics (1978, Issue 4) describes a multi-solvent extraction process using petroleum ether and methanol (Narayanaswami et al, 1978). Smith and Vaughan in Journal of Pharmacy and Pharmacology (1977, 29 (5) discloses the use of various solvents as extraction medium for solubilizing cannabinoids (Smith & Vaughan 1977) [U.S. Pat. No. 7,700,368, which is herein incorporated by reference, describes a purification process for prepare purified forms of cannabinoid and cannabinoid acid constituents of cannabis herb, including the cannabinoid acids Δ9 THCA and CBDA, the corresponding free cannabinoids Δ9 THC and CBD, and the minor cannabinoids, by using a combination of solvent extraction, chromatography and re-crystallization steps (U.S. Pat. No. 7,700,368B2). Furthermore, WO 2004026802, which is herein incorporated by reference, describes a method for preparing cannabidiol from plant material. Substantially pure CBD from plant material is obtained from cannabidiol-containing extract of the plant material by dissolving the extract in a solvent to form a solution, removing insoluble material from this solution and evaporating the solvent from the solution to obtain substantially pure cannabidiol. Furthermore, an example of the purification of cannabidiol is described in Waters Application Notes (Aubin, 2015).

It is preferred to use cannabis plant material derived from cannabis plants having a relatively high content of CBD (as CBDA and/or CBD). For example, by using standard selective breeding techniques cannabis varieties with high CBD content may be developed that may for example have a CBDA/CBD content of >90% of the total cannabinoid content for example Charlotte's Web or Avidekal.

If the plant material from which CBD is to be prepared contains significant amounts of the cannabinoid acid (CBDA) then the plant material may be subjected to a decarboxylation step to convert CBDA to the free cannabinoid CBD. This may be carried out prior to preparation of the CBD-containing plant extract or may form part of this extraction process.

A “substantially pure” preparation of cannabinoid such for example CBD and/or CBG and/or THC is defined as a preparation having a chromatographic purity of 95% or greater, more preferably 96% or greater, more preferably 97% or greater, more preferably 98% or greater, more preferably 99% or greater, and most preferably 99.5% or greater as determined by area normalization ‘of an HPLC UV profile.

A “substantially pure” preparation of cannabinoid such for example CBD and/or ABn-CBD and/or CBG and/or THC can also be commercially purchased either from plant source, microbial source or synthetically produced.

Extraction and Purification of CBD, THC, CBG

The cannabinoids such as CBD, THC, CBG is extracted and purified from high CBD, THC and CBG strains of cannabis respectively, by using the combination of supercritical fluid extraction and chromatography technologies known in the scientific art to a purity of greater than 95%. The CBD, THC and CBG can also be synthesized using chemical synthesis techniques known in the scientific art.

Dried Cannabis buds will be harvested from CBD rich (≥4% w/w, on dry weight basis) cannabinoid strains, THC rich (≥5%w/w, on dry weight basis) cannabinoid strains, and CBG rich (≥1% w/w, on dry weight basis) cannabinoid strains. The dried buds will be extracted separately with a solvent, yielding an extract between 100-300 grams per kilogram of extract per dried buds. The resulting CBD or THC or CBG extract will be passed through a chromatographic column(s) to fractionate CBD or THC or CBG, respectively, out of the extract. The collected CBD rich fractions (>75% CBD w/w) or THC rich fractions (>75% THC w/w) or CBG rich fractions (>75% CBG w/w) will be further separated in a high-pressure column chromatography, to collect pure CBD (purity >98.5%) or THC (purity >98.5%) or CBG (purity >98.5%), respectively.

Examples of extraction method include using 1) organic solvents (toluene and trimethyl pentane), low molecular weight chlorinated hydrocarbon (chloroform and dichloromethane), and/or low molecular weight alcohol (ethanol), or 2) a supercritical fluid (CO2) with or without an organic solvent modifier.

An example of the purification of cannabidiol is described in Waters Application Notes (Aubin, 2015). Briefly, a 500-mg portion of the extract will be sonicated in 10 mL of methanol for 30 minutes and mixed using a magnetic stirrer at 300 rpm. Prior to injection, the sample will be filtered through glass fiber to remove any debris. The preparative chromatographic separation will be carried out using Waters Prep 150 LC System. The CBD will be detected using 2489 UV/Visible detector with semi-prep TaperSlit Flow Cell. The injection volume will be 320 μL. The fraction will be collected using Waters Fraction Collector III. The collected fractions will be pooled and solvent will be evaporated to obtain pure CBD. The method will include appropriate chromatographic system (Prep 150 LC), column temperature (Ambient), flow rate (30.0 mL/min), mobile phase (mobile phase—A: water and mobile phase B—methanol), gradient (85% to 100% B over 2.5 minutes, hold at 100%B for 2 minutes), column (Sunfire C18 OBD™ Prep, 100 Å, 5 μm, 19×100 mm) and detection systems (UV at 228 nm). The example described in Waters Application Notes can be applied to obtain pure THC and pure CBG, with minor method modification known to a skilled artisan. (Aubin, 2015). Analytical chromatographic separations will be carried out using UPLC system equipped with PDA detector. The method will include the appropriate chromatographic system (Waters ACQUITY UPLC H-Class), column (ACQUITY UPLC BEH C18, 130 Å, 175 μm, 2.1×50 mm), column temperature (50° C.), flow rate (1.0 mL/min), mobile phase (mobile phase A—0.1% formic acid in water and mobile phase B—0.1% formic acid in acetonitrile), gradient (60% to 73% B over 2.5 minutes), and detection systems (UV at 228 nm).

The UPLC-determined purity of purified CBD will be >98.5%, THC will be >98.5%, and CBG will be >98.5%.

It will be readily understood by the skilled artisan that numerous alterations may be made to the examples and instructions given herein for the purification and formulation of CBD, THC and CBG.

### Skin Disorders

The compositions or formulations described herein may be used for treating a skin disorder or skin disease. The skin disorder may be but is not limited to an inflammatory skin condition, such as psoriasis, psoriatic arthritis or atopic dermatitis. Some inflammatory skin conditions such as psoriasis may also be classified as autoimmune skin disorders, i.e. caused by a malfunction of the body's immune system.

The compositions and formulations described herein may therefore be used for psoriasis treatment. Psoriasis for example may include plaque psoriasis, guttate psoriasis, inverse psoriasis, pustular psoriasis, and erythrodermic psoriasis.

Accordingly, the formulation described herein may be used to treat plaque psoriasis, guttate psoriasis, inverse psoriasis, pustular psoriasis, erythrodermic psoriasis, psoriatic arthritis, atopic dermatitis or a combination thereof. The psoriasis may be mild, moderate or severe.

‘Plaque psoriasis’ is the most common form of the disease and appears as raised, red patches covered with a silvery white buildup of dead skin cells. These patches or plaques most often show up on the scalp, knees, elbows and lower back. They are often itchy and painful, and they can crack and bleed.

‘Guttate psoriasis’ is a form of psoriasis that appears as small, dot-like lesions. Guttate psoriasis often starts in childhood or young adulthood, and can be triggered by a strep infection. This is the second-most common type of psoriasis, after plaque psoriasis. About 10 percent of people who get psoriasis develop guttate psoriasis.

‘Inverse psoriasis’ shows up as very red lesions in body folds, such as behind the knee, under the arm or in the groin. It may appear smooth and shiny. Many people have another type of psoriasis elsewhere on the body at the same time.

‘Pustular psoriasis’ in characterized by white pustules (blisters of noninfectious pus) surrounded by red skin. The pus consists of white blood cells. It is not an infection, nor is it contagious. Pustular psoriasis can occur on any part of the body, but occurs most often on the hands or feet.

Trythrodermic psoriasis' is a particularly severe form of psoriasis that leads to widespread, fiery redness over most of the body. It can cause severe itching and pain, and make the skin come off in sheets. It is rare, occurring in 3 percent of people who have psoriasis during their life time. It generally appears on people who have unstable plaque psoriasis.

‘Psoriatic arthritis’ is an autoimmune joint disease characterized by both psoriasis and a related form of inflammatory arthritis. It is an aggressive and potentially destructive, inflammatory arthritis. Rarely, a person can have psoriatic arthritis without having obvious psoriasis. Usually, the more severe the skin symptoms are, the greater the likelihood a person will have psoriatic arthritis. Psoriatic arthritis typically affects the large joints, especially those of the lower extremities, distal joints of the fingers and toes, and also can affect the back and sacroiliac joints of the pelvis. For most people, appropriate treatments will relieve pain, protect the joints, and maintain mobility. Physical activity helps maintain joint movement.

‘Atopic dermatitis’ is a chronic, pruritic, inflammatory skin disease that occurs most frequently in children, but also affects many adults. Clinical features of atopic dermatitis include skin dryness, erythema, oozing and crusting, and lichenification. Pruritus is a hallmark of the condition and is responsible for much of the disease burden for patients and their families.

The psoriasis may be related to inflammatory skin conditions, plaques of red skin, often covered with loose, silver-colored scales; itchy and painful lesions, or open wound, scar that sometimes crack and bleed. In severe cases, the plaques of irritated skin will grow and merge into one another, covering large areas. Disorders of the fingernails and toenails, including discoloration and pitting of the nails; the nails may also begin to crumble or detach from the nail bed; plaques of scales or crust on the scalp. Psoriasis can also be associated with psoriatic arthritis, which leads to pain and swelling in the joints. This may be related to eczema or atopic dermatitis, acne, wound, scar and all forms of inflammatory skin conditions, related pain, anxiety and psoriatic arthritis.

### Pharmaceutical Compositions

In another aspect, the present disclosure relates to a pharmaceutical composition or formulation comprising one or more PPAR agonist. The PPAR agonist may comprise one or more compound according to formula I as described herein. The pharmaceutical composition or formulation may further comprise one or more cannabinoid such as CBD or Abn-CBD.

The pharmaceutical composition may comprise one or more physiologically acceptable surface-active agents, carriers, penetrating agents, diluents, excipients, smoothing agents, suspension agents, film forming substances, and coating assistants, or a combination thereof. Acceptable carriers or diluents for therapeutic use are well known in the pharmaceutical art, and are described, for example, in Remington's Pharmaceutical Sciences, 18th Ed., Mack Publishing Co., Easton, Pa. (Remington & Gennaro, 1990), which is incorporated herein by reference in its entirety. Preservatives, stabilizers, dyes, sweeteners, fragrances, flavoring agents, and the like may be provided in the pharmaceutical composition. For example, sodium benzoate, ascorbic acid and esters of p-hydroxybenzoic acid may be added as preservatives. In addition, antioxidants and suspending agents may be used. In various embodiments, alcohols, esters, sulfated aliphatic alcohols, and the like may be used as surface active agents; sucrose, glucose, lactose, starch, crystallized cellulose, mannitol, light anhydrous silicate, magnesium aluminate, magnesium methasilicate aluminate, synthetic aluminum silicate, calcium carbonate, sodium acid carbonate, calcium hydrogen phosphate, calcium carboxymethyl cellulose, and the like may be used as excipients; magnesium stearate, talc, hardened oil and the like may be used as smoothing agents; coconut oil, olive oil, sesame oil, peanut oil, soya may be used as suspension agents or lubricants; cellulose acetate phthalate as a derivative of a carbohydrate such as cellulose or sugar, or methylacetate-methacrylate copolymer as a derivative of polyvinyl may be used as suspension agents; and plasticizers such as ester phthalates and the like may be used as suspension agents.

The term “pharmaceutical composition” refers to a mixture of a compound, formulation or combination of compounds disclosed herein with other chemical components, such as diluents, excipients, penetrating agents or carriers. The pharmaceutical composition facilitates administration of the compound or formulation to an organism. Multiple techniques of administering a compound or formulation exist in the art including, but not limited to, oral, injection, aerosol, parenteral, and topical administration. Pharmaceutical compositions can also be obtained by reacting compounds or formulations with inorganic or organic acids such as hydrochloric acid, hydrobromic acid, sulfuric acid, sulfonic acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound or formulation into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound or formulation.

The term “physiologically acceptable” defines a carrier or diluent that does not abrogate the biological activity and properties of the compound.

The compounds or combinations described herein can be administered to a human patient per se, or in pharmaceutical compositions where they are mixed with suitable carriers, penetrating agents or excipient(s). Techniques for formulation and administration of the compounds of the instant application may be found in “Remington's Pharmaceutical Sciences,” Mack Publishing Co., Easton, Pa., 18th edition, 1990 (Remington & Gennaro, 1990), Suitable routes of administration may, for example, include topical, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections. The compounds or formulation may also be administered in sustained or controlled release dosage forms, including depot injections, osmotic pumps, pills, transdermal (including electrotransport) patches, and the like, for prolonged and/or timed, pulsed administration at a predetermined rate.

The pharmaceutical compositions of the present disclosure may be manufactured in a manner that is itself known, e.g., by means of conventional mixing, dissolving, granulating, dragee-making, levigating, emulsifying, encapsulating, entrapping or tableting processes.

Pharmaceutical compositions for use in accordance with the present disclosure thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above (Remington & Gennaro, 1990).

Injectables can be prepared in conventional forms, either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride, and the like. In addition, if desired, the injectable pharmaceutical compositions may contain minor amounts of nontoxic auxiliary substances, such as wetting agents, pH buffering agents, and the like. Physiologically compatible buffers include, but are not limited to, Hanks's solution, Ringer's solution, or physiological saline buffer. If desired, absorption enhancing preparations (for example, liposomes), may be utilized.

For transmucosal administration, penetrants appropriate to the barrier to be permeated may be used in the formulation.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the compounds can be formulated readily by combining the active compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a patient to be treated. Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragees coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

### Methods of Treatment

The formulation or pharmaceutical preparations as described herein may be used to treat skin disorders like psoriasis, psoriatic arthritis or atopic dermatitis or related skin diseases in a subject. Therefore, the current disclosure also provides a method of treating skin disorders. The skin disorders may be psoriasis, psoriatic arthritis or atopic dermatitis or related skin diseases and may be treated by administering to a subject in need thereof a formulation or pharmaceutical preparations as described herein.

The treating may comprise a treatment period of certain days and can be extended. The treating may comprise a protracted treatment period. For example, the treatment period may be less than 60 days. For example, the treatment period may be between about 1 to about 60 days, or any number of days therebetween. For example, the treatment period may be 1 day, 2 days, 3 days, 4 day, 5 days, 6 days, 7 days, 8 days, 9 days, 10 days, 12 days, 15 days, 18 days, 21 days, 24 days, 28 days, 35 days, 42 days, 49 days, 56 days, 60 days or any period therebetween. Furthermore, the treating period may be at least 1-7 days, 14 days, 28 days, 35 days, 42 days, 60 days or any time therebetween. Furthermore, the treating may comprise an extended treatment period which may be at least 1 month, 2 months, 3 months, 4 months, 5 months, 6 months, 12 months or more.

The formulation may be administered to a subject topically, orally, intradermally, intranasally, intramusclarly, intraperitoneally, intravenously, or subcutaneously. More specifically, the formulation described herewith may be administered topically, transdermally, transmucosally or orally. The transmucosal surface may be the sublingual, buccal, nasal, ocular, vaginal, and/or rectal mucosae.

The “pharmaceutical composition” mentioned herein can be used or delivered as a topical cream, salve, ointment, solution, suspension, lotion, paste, gel, hydrogel, aerosol, spray, foam, solid, tablet, oil or other topical formulation with drug stabilizers/additives; and/or by using delivery devices such as bandages, patches, microneedles, and/or the like; which may be created or formed and used to administer the “pharmaceutical composition” mentioned herein. As used herein, the terms “gel” or “gel-like” can be used interchangeably.

The formulation may be administered topically, transdermally, transmucosally or orally. The transmucosal surface is the sublingual, buccal, nasal, ocular, vaginal, and/or rectal mucosae. The formulation may be delivered in an appropriate aerosol, liquid, gel, or tablet/solid drug carrier with drug stabilizers/additives.

It is further provided a pharmaceutical preparation for treating psoriasis comprising the formulation as described above and physiologically acceptable surface-active agents, carriers, diluents, excipients, smoothing agents, suspension agents, film forming substances, and coating assistants, or a combination thereof.

### Preparation of Formulations

The topical formulations of the disclosure comprise a topical vehicle suitable for administration to subjects' skin, and an amount of the active ingredients in a concentration effective to prevent or reduce, inhibit or eliminate existing or potential skin conditions.

The active ingredient's concentrations can also be expressed in weight/volume or weight/weight percentage terms which will vary somewhat depending on the density of the vehicle and other components in the formulation.

The active ingredient components are typically incorporated into the present formulations by mixing an appropriate amount of API's into the chosen formulation vehicle, along with such other skin care components as are desired. From a formulation standpoint, it is preferred that the selected API's are sufficiently soluble in the formulation vehicle as to allow a consistent formulation having the desired physical and topical application characteristics.

Suitable topical vehicles and vehicle components for use with the formulations of the disclosure are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol (Dow Corning); hydrocarbon-based materials such as petrolatum and squalane; anionic, cationic and amphoteric surfactants and soaps; sustained-release vehicles such as microsponges and polymer matrices; stabilizing and suspending agents; emulsifying agents; and other vehicles and vehicle components that are suitable for administration to the skin, as well as mixtures of topical vehicle components as identified above or otherwise known to the art. The vehicle may further include components adapted to improve the stability or effectiveness of the applied formulation, such as preservatives, antioxidants, skin penetration enhancers, sustained release materials, and the like. Examples of such vehicles and vehicle components are well known in the art and are described in such reference works as Martindale—The Extra Pharmacopoeia (Pharmaceutical Press, London 1993) and Martin (ed.), Remington's Pharmaceutical Sciences (Remington & Gennaro, 1990); Reynolds et al, 1993).

The choice of a suitable vehicle will depend on the particular physical form and mode of delivery that the formulation is to achieve. Examples of suitable forms include liquids (e.g., gargles and mouthwashes, suspensions, emulsions and the like); solids and semisolids such as gels, foams, pastes, capsules, creams, ointments, “sticks” (as in lipsticks or underarm deodorant sticks), powders and the like; formulations containing liposomes or other delivery vesicles; rectal or vaginal suppositories, creams, foams, gels or ointments; and other forms. Typical modes of delivery include application using the fingers; application using a physical applicator such as a cloth, tissue, swab, stick or brush (as achieved for example by soaking the applicator with the formulation just prior to application, or by applying or adhering a prepared applicator already containing the formulation—such as a treated or premoistened bandage, wipe, washcloth or stick—to the skin); spraying (including mist, aerosol or foam spraying); dropper application (as for example with ear drops); sprinkling (as with a suitable powder form of the formulation); and soaking.

The topical formulations of the present disclosure may be prepared in a variety of physical forms. The primary product forms are solids, creams, lotions, gels/serums, and aqueous liquids. The principal differences between these forms are their physical appearance and viscosity (or thickness), which are governed primarily by the presence and amount of emulsifiers and viscosity adjusters; in fact, the main ingredients are, in many cases, common among these product forms. Moreover, a particular topical formulation may often be prepared in a variety of these forms. Solids are generally firm and non-pourable and commonly are formulated as a bar or stick, or in particulate form; solids may be opaque or transparent, and optionally may contain solvents (including water and alcohol), emulsifiers, moisturizers, emollients, fragrances, dyes/colorants, preservatives and active ingredients. Creams and lotions are often similar to one another, differing mainly in their viscosity (creams are typically thicker and more viscous than lotions); both lotions and creams may be opaque, translucent or clear and often contain emulsifiers, solvents (including water and alcohol) and viscosity adjusting agents. Lotions and creams also may optionally contain moisturizers and emollients (especially in the case of skin care products), as well as fragrances, dyes/colorants, preservatives and active ingredients. Gels/serums may be prepared with a range of viscosities, from thick (high viscosity) to thin (low viscosity) and differ principally from lotions and creams in that gels/serums are usually clear rather than opaque. Like lotions and creams, gels/serums often contain emulsifiers, solvents (including water and alcohol) and viscosity adjusters, and may also contain moisturizers and emollients, fragrances, dyes/colorants, preservatives and active ingredients. Aqueous liquids are thinner than creams, lotions or gels, and are generally transparent; liquids usually do not contain emulsifiers. Liquid topical products often contain other solvents in addition to water (including alcohol) and may also contain viscosity adjusters, moisturizers and emollients, fragrances, dyes/colorants/pigments, preservatives and active ingredients.

Suitable emulsifiers for use in the formulations of the present disclosure include, but are not limited to, Incroquat Behenyl TMS (behentrimonium methosulfate, cetearyl alcohol), non-ionic emulsifiers like polyoxyethylene oleyl ether, PEG40 stearate, ceteareth-12 (e.g., Eumulgin B-1 manufactured by Henkel), ceteareth-20 (e.g., Eumulgin B-2 manufactured by Henkel), ceteareth-30, Lanette 0 (manufactured by Henkel; ceteareth alcohol), glyceryl stearate (e.g., Cutina GMS manufactured by Henkel), PEG-100 stearate, Arlacel 165 (glyceryl stearate and PEG-100 stearate), steareth-2 and steareth-20, or combinations/mixtures thereof, as well as cationic emulsifiers like stearamidopropyl dimethylamine and behentrimonium methosulfate, or combinations/mixtures thereof.

Suitable viscosity adjusting agents (i.e., thickening and thinning agents) for use in the formulations of the present disclosure include, but are not limited to, protective colloids or non-ionic gums such as hydroxyethylcellulose (e.g., Cellosize HEC QP52,000-H, manufactured by Amerchol), xanthan gum, and sclerotium gum (Amigel 1.0), as well as magnesium aluminum silicate (Veegum Ultra), silica, microcrystalline wax, beeswax, paraffin, and cetyl palmitate. In addition, appropriate combinations or mixtures of these vicosity adjusters may be utilized according to the present invention.

Suitable solvents for use in the formulations of the present disclosure include, but are not limited to, water, ethanol, butylene glycol, propylene glycol, isopropyl alcohol, isoprene glycol, glycerin, Carbowax 200, Carbowax 400, Carbowax 600, and Carbowax 800. In addition, combinations or mixtures of these solvents may be used according to the present invention.

Suitable surfactants for use in the formulations of the present disclosure include, but are not limited to, nonionic surfactants like Surfactant 190 (dimethicone copolyol), Polysorbate 20 (Tween 20), Polysorbate 40 (Tween 40), Polysorbate 60 (Tween 60), Polysorbate 80 (Tween 80), lauramide DEA, cocamide DEA, and cocamide MEA, amphoteric surfactants like oleyl betaine and cocamidopropyl betaine (Velvetex BK-35), and cationic surfactants like Phospholipid PTC (Cocamidopropyl phosphatidyl PG-dimonium chloride). Appropriate combinations or mixtures of such surfactants may also be used according to the present invention.

Suitable preservatives for use in the formulations of the present disclosure include, but are not limited to, anti-microbials such as Germaben II (manufactured by ICI; propylene glycol, diazolidinyl urea, methylparaben, and propylparaben), methylparaben, propylparaben, imidazolidinyl urea, benzyl alcohol, sorbic acid, benzoic acid, sodium benzoate, dichlorobenzyl alcohol, and formaldehyde, as well as physical stabilizers and anti-oxidants such as alpha-tocopherol (vitamin E), sodium ascorbate/ascorbic acid, resveratrol, ascorbyl palmitate and propyl gallate. In addition, combinations or mixtures of these preservatives may also be used in the formulations of the present invention.

Suitable moisturizers for use in the formulations of the present disclosure include, but are not limited to, lactic acid and other hydroxy acids and their salts, glycerin, propylene glycol, butylene glycol, sodium PCA, Carbowax 200, Carbowax 400, and Carbowax 800. Suitable emollients for use in the formulations of the present disclosure include, but are not limited to, PPG-15 stearyl ether, lanolin alcohol, lanolin, lanolin derivatives, cholesterol, petrolatum, isostearyl neopentanoate, octyl stearate, mineral oil, isocetyl stearate, Ceraphyl 424 (myristyl myristate), octyl dodecanol, dimethicone (Dow Corning 200-100 cps), phenyl trimethicone (Dow Corning 556), Dow Corning 1401 (cyclomethicone and dimethiconol), and cyclomethicone (Dow Corning 344), and Miglyol 840 (manufactured by Huls; propylene glycol dicaprylate/dicaprate). In addition, appropriate combinations and mixtures of any of these moisturizing agents and emollients may be used in accordance with the present invention.

Suitable fragrances and colors, such as, but not limited to, FD&C Red No. 40 and FD&C Yellow No. 5, may be used in the formulations of the present invention. Other examples of fragrances and colors suitable for use in topical products are known in the art.

Other suitable additional and adjunct ingredients which may be included in the formulations of the present disclosure include, but are not limited to, abrasives, absorbents, anti-caking agents, anti-foaming agents, anti-static agents, astringents (e.g., witch hazel, alcohol, and herbal extracts such as chamomile extract), binders/excipients, buffering agents, chelating agents (e.g., Versene EDTA), film forming agents, conditioning agents, opacifying agents, pH adjusters (e.g., citric acid and sodium hydroxide), and protectants. Examples of each of these ingredients, as well as examples of other suitable ingredients in topical product formulations, may be found in publications by The Cosmetic, Toiletry, and Fragrance Association (CTFA) (Wenninger & McEwen, Jr., 1992).

Also, a variety of product types, including particularly cosmetics, may be formulated in each of the forms described above (i.e., solids, creams, lotions, gels, and liquids). For example, cleansers (for face and body), shampoos/conditioners, hair treatments/dyes/perms/straighteners, antiperspirants/deodorants, make-up products, and other facial, hand and body products may be formulated in any of the five major product forms: solids, creams, lotions, gels, or liquids. Common solid form products include cosmetics such as lipsticks, blushes and rouges, makeup products, antiperspirant and deodorant sticks, and cleansers such as bar soap and powder detergents. Other examples of solid form products include lozenges and suppositories. Common cream and lotion form products include alpha hydroxy acid (AHA) products, moisturizing products and sunscreens, shampoos/conditioners and other hair care products, and cosmetics like concealers and foundations. Common gel products include shaving gels and aftershaves. Common liquid form products include anti-acne solutions, perfumes/colognes, aftershaves, gargles/mouthwashes, and toners/bracers/skin conditioners.

The formulations of the disclosure are most preferably formulated such that the component of the formulation is substantially invisible upon application to the skin. This is particularly true in the case of many cosmetic formulations that are applied to the face or other exposed parts of the body. It will be recognized that in some cases, particularly with colored facial skin care products such as blushes, blemish covers, lipsticks and the like, the formulation will be designed to be visible on the skin.

Other methodologies and materials for preparing formulations in a variety of forms are also described in Anthony L. L. Hunting (ed.), “A Formulary of Cosmetic Preparations (Vol. 2)—Creams, Lotions and Milks,” Micelle Press (England, N.J. 1993). See, for example, Chapter 7, pp. 5-14 (oils and gels); Chapter 8, pp. 15-98 (bases and emulsions); Chapter 9, pp. 101-120 (“all-purpose products”); Chapter 10, pp. 121-184 (cleansing masks, creams, lotions); Chapter 11, pp. 185-208 (foundation, vanishing and day creams); Chapter 12, pp. 209-254 (emollients); Chapter 13, pp. 297-324 (facial treatment products); Chapter 14, pp. 325-380 (hand products); Chapter 15, pp. 381-460 (body and skin creams and lotions); and Chapter 16, pp. 461-484 (baby products); the contents of which are incorporated herein by reference (Hunting 1993).

### Penetration Enhancer

The formulation may further comprise a penetration enhancer, which aids in the delivery of the formulation. The terms “penetrating agent”, “penetration enhancer”, “permeation enhancer” or “permeation enhancer composition” define a compound or composition that facilitates the transport and/or release of the formulation of the present disclosure to the targeted tissues or organs, for example the penetration enhancer may lead to a faster rate of release of the formulation, by increasing the flux of the formulation to and/or through the skin or mucosae.

Permeation-enhancers for use in the formulations of the present disclosure include, but not limited to, vegetable oil composition containing at least one vegetable oil selected from the group consisting of almond oil, babassu oil, castor oil, Clark A oil, coconut oil, corn oil, cotton seed oil, jojoba oil, linseed oil, mustard oil, olive oil, palm oil, peanut oil, macadamia oil, shea oil, safflower oil, sesame oil, soybean oil, sunflower-seed oil and wheat germ oil. Accordingly, preferred vegetable oils within the aforementioned group include coconut oil, corn oil, shea oil, jojoba oil, linseed oil, olive oil, palm oil, peanut oil, safflower oil and soybean oil. With most drugs, mixtures of vegetable oils appear to be preferred rather than individual vegetable oils, and a particularly preferred vegetable oil composition for use herein is a mixture of coconut oil and jojoba oil.

Furthermore, the penetration enhancer may be an oil-based emulsifiers or fatty acid, such as Linoleic acids. In a preferred embodiment, the penetration enhancer is jojoba oil.

The formulation contains penetration enhancers from a group of oils. The oil may be from plant oil, the plant oil may be jojoba oil. The effective amount of jojoba oil may be between about 0.001 to about 50 mg or any amount there between.

Jojoba oil may be obtained from the seeds of the desert shrub Simmonsia chinensis, and it is a mixture of mono esters composed principally of both long chain monounsaturated alcohols and carboxylic acids (Miwa & Spencer, 1976).

These and other objects and features of the present disclosure will be made apparent from the following examples. The following examples, as described, are not intended to be construed as limiting the scope of the present invention.

### EXAMPLES

### Example 1

### Synthesis of PPAR Agonist Compounds

### Scheme I: Synthesis of Benzopyrazole(Indazole)-Acetic Acid Derivatives as PPAR Agonists:

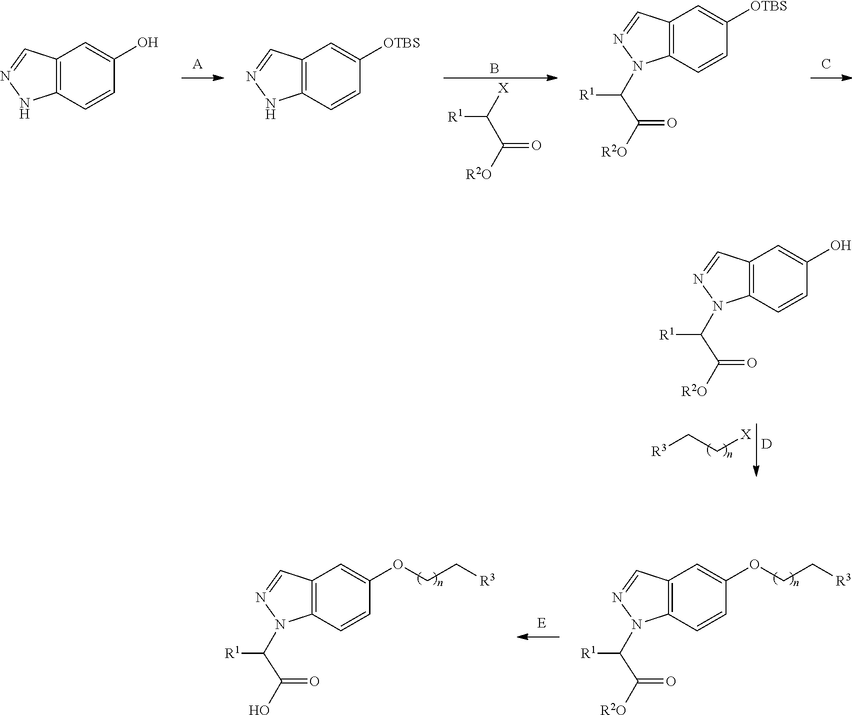

n=1 or 2; R¹=H or —OC₂H₅; R²=CH₃ or C₂H₅; X=Cl, Br

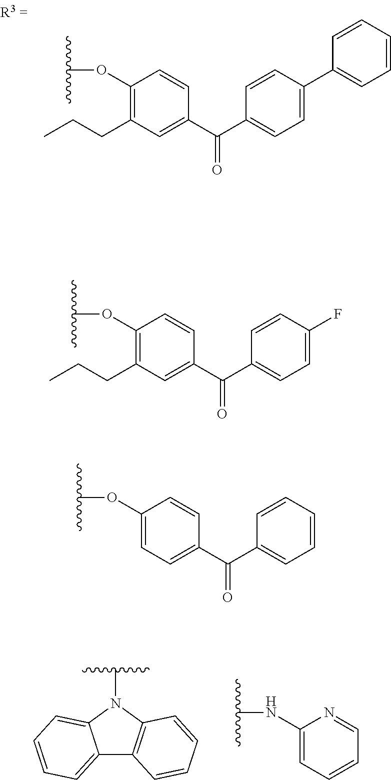 [SMILES: *Oc1ccc(C(=O)c2ccc(-c3ccccc3)cc2)cc1CCC ; *Oc1ccc(C(=O)c2ccc(F)cc2)cc1CCC ; *Oc1ccc(C(=O)c2ccccc2)cc1 ; *n1c2ccccc2c2ccccc21 ; *Nc1ccccn1]

Reagents and conditions: A) TBDMS-C1, imidazole, DMF, r.t, 24 h; B) KtOBu, methyl-2-bromoacetate/ethyl 2-chloro-2-ethoxyacetate, THF, 0° C.—r.t, 0.5 h; C) Methanol in HCl, r.t, 5 h; D) haloalkane, K₂CO₃, cat. KI, ACN, 80° C., 12 h; E) LiOH, THF/H2O (2:1), reflux, 2 h.
Synthesis of 5-(tert-butyl-dimethyl-silanyloxy)-1H-indazole (Procedure A):

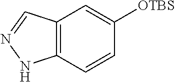 [SMILES: CC(C)(C)[Si](C)(C)Oc1ccc2[nH]ncc2c1]

In a round-bottomed flask, 5-hydroxy-1H-indazole (7.00 g, 52.2 mmol) was dissolved in DMF (100 mL) then TBDMS-Cl (8.65 g, 57.4 mmol) and imidazole (4.26 g, 62.6 mmol) were added. The reaction mixture was stirred at room temperature for 24 h then the solvent was removed in vacuo. The residue was dissolved in ethyl acetate (500 mL), washed twice with water (2×100 mL) followed by brine (100 mL) then dried over sodium sulfate. The organic solvent was evaporated under vacuum and the residue was chromatographed over silica gel with hexane: EtOAc (gradient 0-10% EtOAc) to afford the target compound (12.1 g, 93%) as an off-white solid.

¹H NMR (400 MHz, acetone-d6): δ 0.21 (s, 6H), 1.00 (s, 9H), 6.95 (dd, J=2.1, 8.1 Hz, 1H), 7.17 (d, J=2.1 Hz, 1H), 7.45 (d, J=8.1 Hz, 1H),7.90 (s, 1H).

### General Procedure for N1-Indazole Alkyl Ester Formation (Procedure B):

To a solution of 5-(tert-butyl-dimethyl-silanyloxy)-1H-indazole (1.0 equiv) in anhydrous THF at 0° C., was added Kt-BuO (3 equiv). The reaction mixture was warmed to room temperatur, stirred for 1 h then the reaction mixture was re-cooled to 0° C. After 15 minutes, alkylating agent (1.5 equiv) was added in a single portion, allowed to warm to ambient temperature and stirred for 0.5 h under the N2 atmosphere. After reaction completion, monitored by TLC, the reaction mixture was quenched with saturated ammonium chloride solution. The mixture was extracted with EtOAc (3×). The combined organic layer was washed with distilled water and brine. The organic layer was then dried over anhydrous Sodium sulfate, and the solvent was evaporated under reduced pressure to give the crude products. The crude products were purified using flash chromatography

Synthesis of methyl 2-(5-((tert-butyldimethylsilypoxy)-1H-indazol-1-yl)acetate:

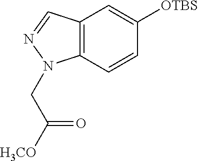 [SMILES: COC(=O)Cn1ncc2cc(O[Si](C)(C)C(C)(C)C)ccc21]

5-((tert-butyldimethylsilyl)oxy)-1H-indazole (5.0 g, 20.1 mmol) was reacted with methyl 2-bromoacetate (4.6 g, 30.2 mmol) according to procedure B to give the crude compound that was purified by flash chromatography using hexane: EtOAc (gradient 0-60% EtOAc) to afford product as a pale-yellow oil (3.1 g, 48% yield). ¹H NMR (400 MHz, acetone-d6): δ 0.23 (s, 6H), 1.00 (s, 9H), 3.71 (s, 3H), 5.26 (s, 2H), 7.01 (dd, J=2.3, 8.7 Hz, 1H), 7.18 (d, J=2.3 Hz, 1H), 7.47 (d, J=8.7 Hz, 1H), 7.91 (s, 1H).

Synthesis of ethyl 2-(5-((tert-butyldimethylsilypoxy)-1H-indazol-1-yl)-2-ethoxyacetate:

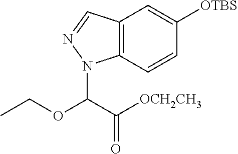 [SMILES: CCOC(=O)C(OCC)n1ncc2cc(O[Si](C)(C)C(C)(C)C)ccc21]

5-((tert-butyldimethylsilyl)oxy)-1H-indazole (5.0 g, 20.1 mmol) was reacted with methyl ethyl 2-chloro-2-ethoxyacetate (5.0 g, 30.2 mmol) according to procedure B to give the crude compound that was purified by flash chromatography using hexane: EtOAc (gradient 0-60% EtOAc) to afford product as a colorless oil (3.1 g, 40%). ¹H NMR (400 MHz, CDCl₃) δ-0.05 (s, 6H), 0.74 (s, 9H), 0.95-0.90 (m, 6H), 3.15-3.29 (m, 1H), 3.33 (dq, J=10.0, 7.0 Hz, 1H), 3.96 (qq, J=7.5, 3.2 Hz, 2H), 5.87 (s, 1H), 6.71 (dd, J=8.9, 2.1 Hz, 1H), 6.83 (d, J=2.2 Hz, 1H), 7.00 (d, J=1.2 Hz, 1H), 7.65 (s, 1H).

Synthesis of methyl 2-(5-hydroxy-1H-indazol-1-yl)acetate:

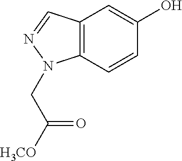 [SMILES: COC(=O)Cn1ncc2cc(O)ccc21]

To a solution of methyl 2-(5-((tert-butyldimethylsilyl)oxy)-1H-indazol-1-y0acetate (3 g, 9.3 mmol) in methanol (10 mL), was added 3NHCl (10 mL) in methanol at room temperature, then stirred for 5 h. After reaction completion, monitored by TLC, solvent was removed under vacuum and residue was purified by flash column chromatography using hexane: EtOAc (gradient 0-50% EtOAc) to afford product as a white solid compound (1.8 g, 90% yield), ¹H NMR (400 MHz, DMSO-d6): δ 3.56 (s, 3H), 5.30 (s, 2H), 6.92 (dd, J=2.3, 8.7 Hz, 1H), 7.0 (d, J=2.3 Hz, 1H), 7.44 (d, J=8.7 Hz, 1H), 7.88 (s, 1H), 9.20 (s, 1H).

Synthesis of methyl 2-ethoxy-2-(5-hydroxy-1H-indazol-1-yl)acetate:

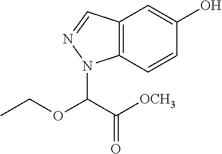 [SMILES: CCOC(C(=O)OC)n1ncc2cc(O)ccc21]

To a solution of ethyl 2-(5-((tert-butyldimethylsilyl)oxy)-1H-indazol-1-yl)-2-ethoxyacetate (3.0 g, 7.9 mmol) in methanol (10 mL), was added 3N HCl in methanol (5 mL) at room temperature, then stirred for 5 h. After reaction completion, monitored by TLC, solvent was removed under vacuum and residue was purified by flash column chromatography using hexane: EtOAc (gradient 0-50% EtOAc) to afford product as a white solid compound (1.7 g, 85% yield). ¹H NMR (400 MHz, CDCl₃) δ 1.19 (t, J=7.0 Hz, 3H), 3.34-3.49 (m, 1H), 3.59 (dq, J=9.6, 7.0 Hz, 1H), 3.76 (s, 3H), 6.16 (s, 1H), 7.02 (dd, J=8.9, 2.3 Hz, 1H), 7.08 (d, J=2.3 Hz, 1H), 7.55 (d, J=8.9 Hz, 1H), 7.91 (s, 1H).

### Scheme II: Synthesis of Substituted Aryl of R³ Derivatives

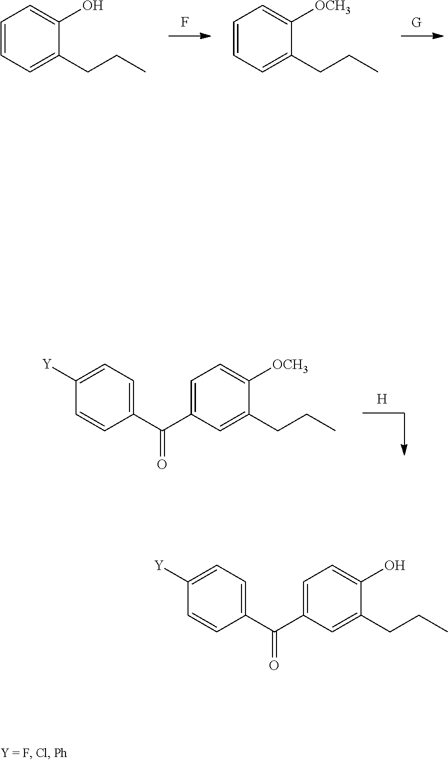

Reagent and conditions: F) CH₃, K₂CO₃, ACN, 40° C., 16 h; G) AlCl₃, Y—C₆H₄—COCl, DCM, 0° C. to rt, 3 h; H) BBr₃, DCM, 0° C. to rt, 5 h

Synthesis of 1-methoxy-2-propyl benzene:

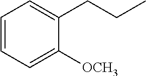

To a mixture of 2-propyl-phenol (10.0 g, 73.4 mmol) and potassium carbonate (20. 2 g, 146.2 mmol) in acetonitrile (150 mL), methyl iodide (31.0 g, 218.4 mmol) was added then the reaction was heated at 40° C. for 16 h. The mixture was cooled to room temperature and filtered to remove suspended salts. The solvent was removed in vacuo and residue was partitioned between ethyl acetate (300 mL) and 1N HCl (60 mL). The organic layer was washed with water (100 mL) followed by brine (100 mL) and then dried over anhydrous sodium sulfate. The solvent was removed in vacuo and the residue was chromatographed over silica gel eluting with hexane: EtOAc (gradient 0-5% EtOAc) to give compound (11.0 g, quantitative yield) as a colorless oil.

¹H NMR (400 MHz, acetone-d6) δ 0.95 (t, J=7.6 Hz, 3H), 1.60 (sext, J=7.6 Hz, 2H), 2.58 (t, J=7.6 Hz, 2H), 3.81 (s, 3H), 6.82-6.90 (m, 2H), 7.11-7.19 (m, 2H)

Synthesis of [1,1′-biphenyl]-4-yl(4-methoxy-3-propylphenyl)methanone:

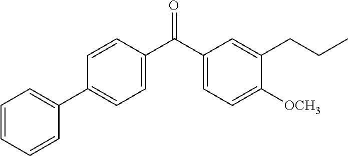 [SMILES: CCCc1cc(C(=O)c2ccc(-c3ccccc3)cc2)ccc1OC]

To a stirred solution of 1-methoxy-2-propylbenzene (6.0 g, 40.0 mmol) and AlCl₃ (8.0 g, 60.0 mmol) in anhydrous DCM (100 mL) maintained at 0° C., was added biphenyl-4-carbonyl chloride (9.53 g, 44.0 mmol) in portions over 15 min under nitrogen atmosphere then the reaction mixture was stirred at room temperature for 3 h. After reaction completion, monitored by TLC, methanol was added to quench the reaction. The solvent was removed in vacuo and the residue was dissolved in ethyl acetate (300 mL), washed with water (2×60 mL) followed by brine (60 mL) then dried over anhydrous sodium sulfate. The solvent was removed in vacuo to give an oily residue which was chromatographed over silica gel eluting with hexane: EtOAc (gradient 0-10% EtOAc) to give the target compound (10.97, 83% yield).

¹H NMR (400 MHz, acetone-d6) δ 0.96 (t, J=7.5 Hz, 3H), 1.62 (sext, J=7.5 Hz, 2H), 2.65 (t, J=7.5 Hz, 2H), 3.96 (s, 3H), 7.10 (d, J=8.4 Hz, 1H), 7.42-7.54 (m, 3H), 7.69-7.79 (m, 6H), 7.85 (d, J=1.5 Hz, 2H).

Synthesis of (4-fluorophenyl)(4-methoxy-3-propylphenyl)methanone:

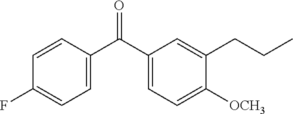 [SMILES: CCCc1cc(C(=O)c2ccc(F)cc2)ccc1OC]

To a stirred solution of 1-methoxy-2-propylbenzene (6.0 g, 40.0 mmol) and AlCl₃ (8.0 g, 60.0 mmol) in anhydrous DCM (100 mL) maintained at 0° C., was added 4-fluorobenzoyl chloride (6.98 g, 44.0 mmol) in portions over 15 min under nitrogen atmosphere then the reaction mixture was stirred at room temperature for 3 h. After reaction completion, monitored by TLC, methanol was added to quench the reaction. The solvent was removed in vacuo and the residue was dissolved in ethyl acetate (300 mL), washed with water (2×60 mL) followed by brine (60 mL) then dried over anhydrous sodium sulfate. The solvent was removed in vacuo to give an oily residue which was chromatographed over silica gel eluting with hexane: EtOAc (gradient 0-10% EtOAc) to give the target compound (9.36 g, 86% yield).

¹H NMR (400 MHz, acetone-d6) δ 0.94 (t, J=7.5 Hz, 3H), 1.57-1.64 (m, 2H), 2.64 (t, J=7.6 Hz, 2H), 3.95 (s, 3H), 7.09 (d, J=8.1 Hz, 1H), 7.28-7.32 (m, 2H), 7.64-7.68 (m, 2H), 7.81-7.85 (m, 2H).

Synthesis of [1,1′-biphenyl]-4-yl(4-hydroxy-3-propylphenyl)methanone:

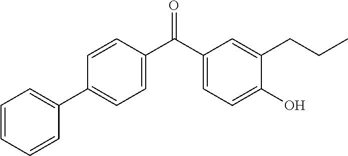 [SMILES: CCCc1cc(C(=O)c2ccc(-c3ccccc3)cc2)ccc1O]

To a stirred solution of [1,1′-biphenyl]-4-yl(4-methoxy-3-propylpheny)methanone (7.0 g, 21.1 mmol) in anhydrous DCM (90 mL) maintained at 0° C. under nitrogen atmosphere, was added BBr₃ (21.2 g, 84.6 mmol) in portion over 15 minutes. The reaction mixture was brought to room temperature and stirred for 5 h. After reaction completion, monitored by TLC, ice cold water was added to quench the reaction. The reaction was partitioned between ethyl acetate (300 mL) and saturated sodium bicarbonate solution (100 mL). The organic layer was separated, washed with water (2×50 mL) followed by brine (50 mL) then dried over anhydrous sodium sulfate. The solvent was removed in vacuo and the residue was chromatographed over silica gel eluting with hexane: EtOAc (gradient 0-30% EtOAc) to afford the target compounds (6.1 g, 91% yield).

¹H NMR (400 MHz, DMSO-d6) δ 0.92 (t, J=7.2 Hz, 3H), 1.57 (sext, J=7.6, 7.2 Hz, 2H), 2.56 (t, J=7.6 Hz, 2H), 5.76 (s, 1H), 6.92 (d, J=8.4 Hz, 1H), 7.42-7.46 (m, 1H), 7.50-7.55 (m, 3H), 7.59 (d, J=2.3 Hz, 1H), 7.76-7.78 (m, 4H), 7.83-785 (m, 2H).

Synthesis of (4-fluorophenyl)(4-hydroxy-3-propylphenyl)methanone:

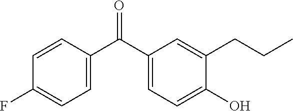 [SMILES: CCCc1cc(C(=O)c2ccc(F)cc2)ccc1O]

To a stirred solution of 4′-fluoro(4-methoxy-3-propyl) benzophenone (7.0 g, 25.7 mmol) in anhydrous DCM (90 mL) maintained at 0° C. under nitrogen atmosphere, was added BBr3 (21.2 g, 84.8 mmol) in portion over 15 minutes. The reaction mixture was brought to room temperature and stirred for 5 h. After reaction completion, monitored by TLC, ice cold water was added to quench the reaction. The reaction was partitioned between ethyl acetate (300 mL) and saturated sodium bicarbonate solution (100 mL). The organic layer was separated, washed with water (2×50 mL) followed by brine (50 mL) then dried over anhydrous sodium sulfate. The solvent was removed in vacuo and the residue was chromatographed over silica gel eluting with hexane: EtOAc (gradient 0-30% EtOAc) to afford the target compounds (5.9 g, 89% yield).

¹H NMR (400 MHz, acetone-d6) δ 0.97 (t, J=7.5 Hz, 3H), 1.60-1.69 (m, 2H), 2.64 (t, J=7.6 Hz, 2H), 6.96 (d, J=8.1 Hz, 1H), 7.21-7.31 (m, 2H), 7.54 (dd, J=2.1, 8.1 Hz, 1H), 7.63 (d, J=2.1 Hz, 1H), 7.80-7.84 (m, 2H).

General Procedure for O/N-Alkylation of Hydroxy/Amino Group of Substituted Aryl (Heteroaryl) Substrates (R³—CH₂—(CH₂)ₙ—X) (Procedure K):

To a solution of substituted aryl/heteroaryl substrates (1.0 equiv) in anhydrous acetonitrile, was added potassium carbonate (3-4 equiv). To the stirring mixture, dibromoalkane (4.0 equiv) and potassium iodide (0.10 equiv) were added, and the reaction was allowed to reflux at 80° C. for 12 h under N₂ atmosphere. After reaction completion, monitored by TLC, the reaction was concentrated in vacuo followed by the addition of 1N HCl to quench the reaction and extracted with EtOAc. The combined organic layers were washed with H₂O and brine and then dried over Sodium sulfate. The solvent was evaporated under reduced pressure and the crude products were purified using flash chromatography.

Synthesis of [1,1′-biphenyl]-4-yl(4-(3-bromopropoxy)-3-propylphenyl)methanone:

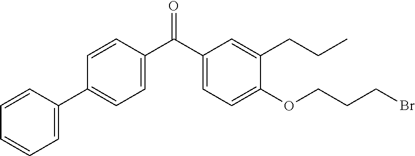 [SMILES: CCCc1cc(C(=O)c2ccc(-c3ccccc3)cc2)ccc1OCCCBr]

A mixture of [1,1′-biphenyl]-4-yl(4-hydroxy-3-propylphenyl)methanone (5.5 g, 17.4 mmol), 1,3-dibromopropane (14.0 g, 69.6 mmol), potassium carbonate (7.2 g, 52.2 mmol), and potassium iodide (0.2 g, 1.7 mmol) were treated according to procedure K to give the crude product that was purified by flash chromatography using hexane: EtOAc (gradient 0-10% EtOAc) to afford product as a white solid (3.8 g, 58% yield).

¹H NMR (400 MHz, CDCl₃) δ 0.96 (dd, J=7.9, 6.7 Hz, 3H), 1.64 (h, J=7.4 Hz, 2H), 2.39 (p, J=6.0 Hz, 2H), 2.64 (t, J=7.6 Hz, 2H), 3.65 (t, J=6.4 Hz, 2H), 4.21 (t, J=5.8 Hz, 2H), 6.83-6.96 (m, 1H), 7.33-7.46 (m, 1H), 7.49 (dd, J=8.3, 6.9 Hz, 2H), 7.59-7.76 (m, 6H), 7.76-7.95 (m, 2H).

Synthesis of [1,1′-biphenyl]-4-yl(4-(2-bromoethoxy)-3-propylphenyl)methanone:

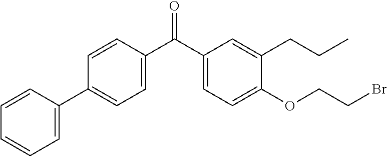 [SMILES: CCCc1cc(C(=O)c2ccc(-c3ccccc3)cc2)ccc1OCCBr]

A mixture of [1,1′-biphenyl]-4-yl(4-hydroxy-3-propylphenyl)methanone (1.0 g, 3.1 mmol), 1,2-dibromoethane (2.3 g, 12.6 mmol), potassium carbonate (1.7 g, 12.4 mmol), and potassium iodide (0.05 g, 0.3 mmol) were treated according to procedure K to give the crude product that was purified by flash chromatography using hexane: EtOAc (gradient 0-10% EtOAc) to afford product as a white solid (0.6 g, 85%). ¹H NMR (400 MHz, CDCl₃) δ 0.97 (t, J=7.4 Hz, 3H), 1.67 (h, J=7.4 Hz, 2H), 2.72-2.64 (m, 2H), 3.71 (t, J=6.1 Hz, 2H), 4.39 (t, J=6.1 Hz, 2H), 6.86 (d, J=8.2 Hz, 1H), 7.44-7.36 (m, 1H), 7.49 (dd, J=8.3, 7.0 Hz, 2H), 7.75-7.63 (m, 6H), 7.85 (d, J=8.1 Hz, 2H).

Synthesis of (4-(3-bromopropoxy)-3-propylphenyl)(4-fluorophenyl)methanone:

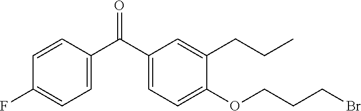 [SMILES: CCCc1cc(C(=O)c2ccc(F)cc2)ccc1OCCCBr]

A mixture of (4-fluorophenyl)(4-hydroxy-3-propylphenyl)methanone (5.0 g, 19.3 mmol), 1,3-dibromopropane (15.6 g, 77.3 mmol), potassium carbonate (10.6 g, 76.7 mmol), and potassium iodide (0.32 g, 1.9 mmol) were treated according to procedure K to give the crude product that was purified by flash chromatography using hexane: EtOAc (gradient 0-10% EtOAc) to afford product as a white solid (3.6 g, 49% yield). ¹H NMR (400 MHz, CDCl₃) δ 0.96-0.93 (m, 3H), 1.50-1.68 (m, 3H), 2.38 (p, J=6.1 Hz, 2H), 2.62 (t, J=7.6 Hz, 1H), 3.64 (t, J=6.4 Hz, 2H), 4.20 (t, J=5.8 Hz, 2H), 6.78-7.07 (m, 1H), 7.05-7.21 (m, 2H), 7.64 (dd, J=7.6, 1.7 Hz, 2H), 7.67-8.01 (m, 2H).

Synthesis of (4-(2-bromoethoxy)-3-propylphenyl)(4-fluorophenyl)methanone:

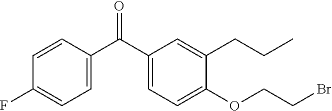 [SMILES: CCCc1cc(C(=O)c2ccc(F)cc2)ccc1OCCBr]

A mixture of (4-fluorophenyl)(4-hydroxy-3-propylphenyl)methanone (1.0 g, 3.8 mmol), 1,2-dibromoethane (2.9 g, 15.5 mmol), potassium carbonate (2.5 g, 15.2 mmol), and potassium iodide (0.06 g, 0.38 mmol) were treated according to procedure K to give the crude product that was purified by flash chromatography using hexane: EtOAc (gradient 0-10% EtOAc) to afford product as a white solid (0.7 g, 50% yield). ¹H NMR (400 MHz, CDCl₃) δ 0.97-0.93 (m,3H), 1.67 (hept, J=7.4 Hz, 2H), 2.47-2.92 (m, 2H), 4.12-4.57 (m, 4H), 6.71-7.04 (m, 1H), 7.08-7.22 (m, 2H), 7.46-7.74 (m, 1H), 7.71-7.92 (m, 2H), 8.05 (t, J=1.7 Hz, 1H).

Synthesis of 1-(3-bromopropoxy)-3-phenoxybenzene:

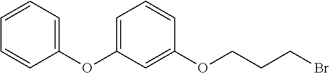 [SMILES: BrCCCOc1cccc(Oc2ccccc2)c1]

A mixture of 3-phenoxyphenol (0.5 g, 2.7 mmol), 1,3-dibromopropane (2.18 g, 10.74 mmol), potassium carbonate (1.1 g, 8.1 mmol), and potassium iodide (0.05 g, 0.27 mmol) were treated according to procedure K to give the crude product that was purified by flash chromatography using hexane: EtOAc (gradient 0-10% EtOAc) to afford oily product (0.54 g, 65% yield).

¹H NMR (400 MHz, DMSO) δ 2.25 (p, J=6.2 Hz, 2H), 3.68 (t, J=6.5 Hz, 2H), 4.07 J=6.0 Hz, 2H), 6.88-6.95 (m, 2H), 7.07 (td, J=7.3, 1.1 Hz, 1H), 7.30-7.39 (m, 2H).

Synthesis of 9-(2-bromoethyl)-9H-carbazole:

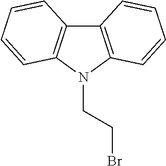 [SMILES: BrCCn1c2ccccc2c2ccccc21]

A mixture of 9H-carbazole (1.0 g, 6.0 mmol), 1,2-dibromoethane (4.8 g, 24.0 mmol), potassium carbonate (3.3 g, 24.0 mmol), and potassium iodide (0.09 g, 0.6 mmol) were treated according to procedure K to give the crude product that was purified by flash chromatography using hexane: EtOAc (gradient 0-10% EtOAc) to afford oily product (54% yield).

¹H NMR (400 MHz, DMSO) δ 3.92 (t, J=6.3 Hz, 2H), 4.86 (t, J=6.3 Hz, 2H), 7.18-7.27 (m, 2H), 7.46 (ddd, J=8.3, 7.2, 1.2 Hz, 2H), 7.67 (d, J=8.2 Hz, 2H), 8.16 (dt, J=7.7, 0.9 Hz, 2H).

### General Procedure for O-Alkylation of N′-Indazole Alkyl Ester Component (Procedure D):

To a solution of N¹-indazole alkyl ester component (1.0 equiv) in dry acetonitrile, was added potassium carbonate (3.0 equiv), halo alkyl derivative of aryl/heteroaryl ether/amine (1.10 equiv), and potassium iodide (0.10 equiv). The reaction was allowed to reflux at 80° C. for 12 h under N₂ atmosphere. After reaction completion, monitored by TLC, the reaction was concentrated in vacuo followed by the addition of 1N HCl to quench the reaction and extracted with EtOAc. The combined organic layers were washed with H₂O and brine and then dried over Sodium sulfate. The solvent was evaporated under reduced pressure and the crude products were purified using flash chromatography.

Synthesis of methyl 2-(5-(3-(4-([1,1′-biphenyl]-4-carbonyl)-2-propylphenoxy)propoxy)-1H-indazol-1-yl)acetate—Compound “MP-153”:

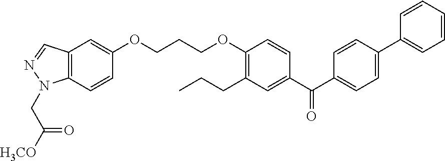 [SMILES: CCCc1cc(C(=O)c2ccc(-c3ccccc3)cc2)ccc1OCCCOc1ccc2c(cnn2CC(=O)OC)c1]

A mixture of methyl 2-(5-hydroxy-1H-indazol-1-yl)acetate (2.0 g, 9.7 mmol), [1,1′-biphenyl]-4-yl(4-(3-bromopropoxy)-3-propylphenyl)methanone, (4.6 g, 10.6 mmol), potassium carbonate (4.0 g, 29.1 mmol), and potassium iodide (0.16 g, 0.97 mmol) were treated according to procedure D to give the crude compound that was purified by flash chromatography using DCM: EtOAc (gradient 2-20% EtOAc) to afford the target compound (3.5 g, 64% yield) as a white solid.

¹H NMR (400 MHz, DMSO-d6) δ 0.88 (t, J=7.6 Hz, 3 H), 1.57 (sext, J=7.6 Hz, 2H), 2.28 (quin, J=6.4 Hz, 2 H), 2.62 (t, J=7.6 Hz, 2 H), 3.66 (s, 3H), 4.22 (t, J=6.4 Hz, 2H), 4.30 (t, J=6.4 Hz, 2H), 5.36 (s, 2 H), 7.08 (dd, J=2.3, 8.7 Hz, 1H), 7.16 (d, J=8.7 Hz, 1H), 7.24 (d, J=2.3 Hz, 1H), 6.42-6.46 (m, 1H), 7.50-7.57 (m, 3H), 7.63-7.66 (m, 2H), 7.76-7.79 (m, 4H), 7.84-7.86 (m, 2H), 7.98 (s, 1H).

Synthesis of methyl 2-(5-(3-(4-(4-fluorobenzoyl)-2-propylphenoxy)nropoxy)-1H-indazol-1-yl)acetate—compound “MP-152”:

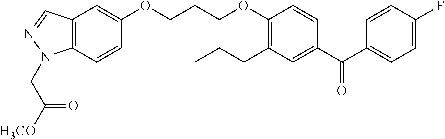 [SMILES: CCCc1cc(C(=O)c2ccc(F)cc2)ccc1OCCCOc1ccc2c(cnn2CC(=O)OC)c1]

A mixture of methyl 2-(5-hydroxy-1H-indazol-1-yl)acetate (2.0 g, 9.7 mmol), (4-(3-bromopropoxy)-3-propylphenyl)(4-fluorophenyl)methanone, (4.0 g, 10.6 mmol), potassium carbonate (4.0 g, 29.1 mmol), and potassium iodide (0.16 g, 0.97 mmol) were treated according to procedure D to give the crude compound that was purified by flash chromatography using hexane: EtOAc (gradient 0-30% EtOAc) to afford the product as a white solid (3.5 g, 72% yield).

¹H NMR (400 MHz, DMSO-d6) δ 0.87 (t, J=7.2 Hz, 3H), 1.52-1.59 (m, 2H), 2.25-2.30 (m, 2H), 2.58-2.62 (m, 2H), 3.66 (s, 3H), 4.16 (d, J=5.9 Hz, 2H), 4.21 (t, J=5.9 Hz, 2H), 4.29 (t, J=5.9 Hz, 2H), 5.36 (s, 2 H), 7.07 (dd, J=2.4, 8.5 Hz, 1H), 7.15 (d, J=8.5 Hz, 1H), 7.23 (d, J=2.4 Hz, 1H), 7.35-7.40 (m, 2H), 7.55-7.61 (m, 3H), 7.75-7.79 (m, 2H), 7.97 (s, 1H).

Synthesis of methyl 2-(5-(3-(4-phenoxyphenoxy)propoxy)-1H-indazol-1-yl)acetate—Compound “MH-97”:

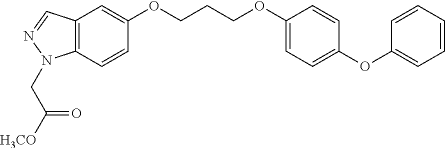 [SMILES: COC(=O)Cn1ncc2cc(OCCCOc3ccc(Oc4ccccc4)cc3)ccc21]

A mixture of methyl 2-(5-hydroxy-1H-indazol-1-yl)acetate (0.2 g, 0.97 mmol), 1-(3-bromopropoxy)-4-phenoxybenzene (0.33 g, 1.07 mmol), potassium carbonate (0.4 g, 2.9 mmol), and potassium iodide (0.016 g, 0.01 mmol) were treated according to procedure D to give the crude compound that was purified by flash chromatography using hexane: EtOAc (gradient 0-30% EtOAc) to afford the product as a white solid (0.29 g, 46% yield).

¹H NMR (400 MHz, DMSO) δ 2.21 (p, J=6.1 Hz, 2H), 3.67 (s, 3H), 4.16 (q, J=6.5 Hz, 4H), 5.36 (s, 2H), 6.91 (s, 1H), 6.91-7.07 (m, 6H), 7.05-7.12 (m, 2H), 7.24 (d, J=2.2 Hz, 1H), 7.34 (t, J=7.4 Hz, 2H), 7.56 (d, J=9.0 Hz, 1H), 7.97 (s, 1H).

Synthesis of methyl 2-(5-(2-(9H-carbazol-9-yl)ethoxy)-1H-indazol-1-yl)acetate—Compound “MH-104”:

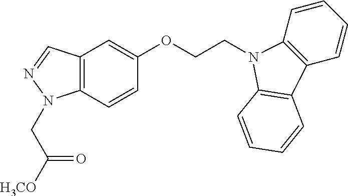 [SMILES: COC(=O)Cn1ncc2cc(OCCn3c4ccccc4c4ccccc43)ccc21]

A mixture of methyl 2-(5-hydroxy-1H-indazol-1-yl)acetate (0.2 g, 0.97 mmol), 9-(2-bromoethyl)-9H-carbazole (0.29 g, 1.06 mmol), potassium carbonate (0.4 g, 2.9 mmol), and potassium iodide (0.016 g, 0.01 mmol) were treated according to procedure D to give the crude compound that was purified by flash chromatography using hexane: EtOAc (gradient 0-30% EtOAc) to afford the product as a white solid (0.15 g, 41% yield).

¹H NMR (400 MHz, DMSO) δ 3.64 (s, 3H), 4.40 (t, J=5.3 Hz, 2H), 4.85 (t, J=5.3 Hz, 2H), 5.32 (s, 2H), 6.85 (dd, J=9.1, 2.3 Hz, 1H), 7.16 (d, J=2.3 Hz, 1H), 7.22 (t, J=7.5 Hz, 2H), 7.48 (t, J=7.8 Hz, 3H), 7.72 (d, J=8.2 Hz, 2H), 7.92 (s, 1H), 8.16 (d, J=7.7 Hz, 2H).

Synthesis of methyl 2-ethoxy-2-(5-(3-(4-(4-fluorobenzoyl)-2 propylphenoxy)propoxy)-1H-indazol-1-yl)acetate—Compound “KV-157”:

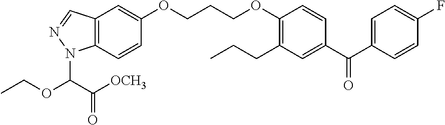 [SMILES: CCCc1cc(C(=O)c2ccc(F)cc2)ccc1OCCCOc1ccc2c(cnn2C(OCC)C(=O)OC)c1]

A mixture of methyl 2-ethoxy-2-(5-hydroxy-1H-indazol-1-yl)acetate (0.1 g, 0.40 mmol), (4-(3-bromopropoxy)-3-propylphenyl)(4-fluorophenyl)methanone (0.16 g, 0.44 mmol), potassium carbonate (0.22 g, 1.60 mmol), and potassium iodide (0.006 g, 0.04 mmol) were treated according to procedure D to give the crude compound that was purified by flash chromatography using hexane: EtOAc (gradient 0-30% EtOAc) to afford the product as a white solid (0.16 g, 73% yield).

¹H NMR (400 MHz, CDCl₃) δ 0.92 (t, J=7.3 Hz, 3H), 1.18 (t, J=7.1 Hz, 3H), 1.53-1.66 (m, 2H), 2.37 (q, J=6.0 Hz, 2H), 2.63 (t, J=7.6 Hz, 2H), 3.43 (dd, J=9.5, 7.0 Hz, 1H), 3.53-3.64 (m, 1H), 3.76 (d, J=1.3 Hz, 3H), 4.19-4.31 (m, 4H), 6.16 (s, 1H), 6.91 (d, J=9.1 Hz, 1H), 7.06-7.18 (m, 4H), 7.55-7.66 (m, 3H), 7.79 (dd, J =8.8, 5.4 Hz, 2H), 7.94 (s, 1H).

Synthesis of ethyl 2-ethoxy-2-(5-(2-(4-(4-fluorobenzoyl)-2-propylphenoxy)ethoxy)-1H-indazol-1-yl)acetate—Compound “KV-163A”:

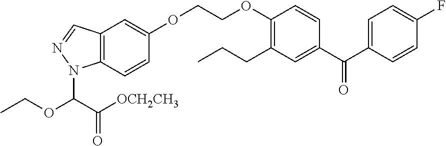 [SMILES: CCCc1cc(C(=O)c2ccc(F)cc2)ccc1OCCOc1ccc2c(cnn2C(OCC)C(=O)OCC)c1]

A mixture of ethyl 2-ethoxy-2-(5-hydroxy-1H-indazol-1-yl)acetate (0.1 g, 0.37 mmol), (4-(2-bromoethoxy)-3-propylphenyl)(4-fluorophenyl)methanone (0.15 g, 0.41 mmol), potassium carbonate (0.15 g, 1.11 mmol), and potassium iodide (0.006 g, 0.03 mmol) were treated according to procedure D to give the crude compound that was purified by flash chromatography using hexane: EtOAc (gradient 0-30% EtOAc) to afford the product as a white solid (0.14 g, 70% yield).

¹H NMR (400 MHz, Acetone)δ 0.88 (t, J=7.3 Hz, 3H), 1.01-1.26 (m, 6H), 1.63 (q, J=7.4 Hz, 2H), 2.65 (t, J=7.5 Hz, 2H), 3.41 (dd, J=9.2, 6.8 Hz, 1H), 3.52-3.68 (m, 1H), 4.05 (q, J=7.1 Hz, 1H), 4.19 (tq, J=7.4, 3.1 Hz, 2H), 4.46-4.61 (m, 4H), 6.27 (d, J=1.3 Hz, 1H), 7.12-7.21 (m, 2H), 7.26-7.36 (m, 3H), 7.62-7.70 (m, 3H), 7.79-7.87 (m, 2H), 7.98 (s, 1H).

Synthesis of ethyl 2-(5-(3-(4-([1,1′-biphenyl]-4-carbonyl)-2-propylphenoxy)propoxy)-1H-indazol-1-yl)-2-ethoxyacetate—Compound “KV-165”:

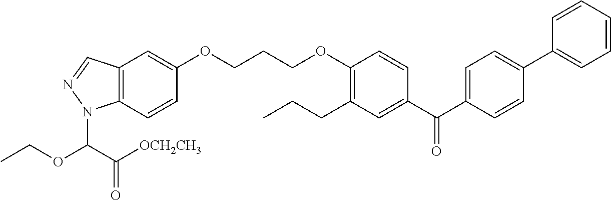 [SMILES: CCCc1cc(C(=O)c2ccc(-c3ccccc3)cc2)ccc1OCCCOc1ccc2c(cnn2C(OCC)C(=O)OCC)c1]

A mixture of ethyl 2-ethoxy-2-(5-hydroxy-1H-indazol-1-yl)acetate (0.1 g, 0.37 mmol), [1,1′-biphenyl]-4-yl(4-(3-bromopropoxy)-3-propylphenyl)methanone (0.17 g, 0.40 mmol), potassium carbonate (0.15 g, 1.13 mmol), and potassium iodide (0.006 g, 0.03 mmol) were treated according to procedure D to give the crude compound that was purified by flash chromatography using hexane: EtOAc (gradient 0-30% EtOAc) to afford the product as a white solid (0.16 g, 72% yield).

¹H NMR (400 MHz, Acetone) δ 0.79 (t, J=7.3 Hz, 3H), 0.91-1.09 (m, 6H), 1.50 (q, J=7.5 Hz, 2H), 2.19-2.35 (m, 2H), 2.56 (t, J=7.6 Hz, 2H), 3.27 (dd, J=9.2, 6.9 Hz, 1H), 3.47 (dd, J=9.0, 6.6 Hz, 1H), 4.06 (dd, J=7.1, 2.9 Hz, 2H), 4.19 (t, J=6.1 Hz, 2H), 4.26 (t, J=6.1 Hz, 2H), 6.13 (s, 1H), 6.95-7.09 (m, 2H), 7.16 (d, J=2.3 Hz, 1H), 7.31 (d, J=7.3 Hz, 1H), 7.35-7.44 (m, 2H), 7.50 (d, J=9.1 Hz, 1H), 7.58 (d, J=9.1 Hz, 2H), 7.60-7.67 (m, 2H), 7.71 (s, 4H), 7.83 (s, 1H).

Synthesis of Ethyl 2-(5-(2-(4-([1,1,′-biphenyl]-4-carbonyl)-2-propylphenoxy)ethoxy)-1H-indazol-1-yl)-2-ethoxyacetate—Compound “KV-194A”:

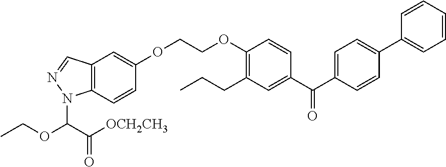 [SMILES: CCCc1cc(C(=O)c2ccc(-c3ccccc3)cc2)ccc1OCCOc1ccc2c(cnn2C(OCC)C(=O)OCC)c1]

A mixture of ethyl 2-ethoxy-2-(5-hydroxy-1H-indazol-1-yl)acetate, (0.1g, 0.37 mmol) [1,1′-biphenyl]-4-yl(4-(2-bromoethoxy)-3-propylphenyl)methanone (0.17 g, 0.40 mmol), potassium carbonate (0.15 g, 1.13 mmol), and potassium iodide (0.006 g, 0.03 mmol) were treated according to procedure D to give the crude compound and was purified by flash chromatography using hexane: EtOAc (gradient 0-30% EtOAc) to afford the product as a white solid (0.13 g, 59% yield).

¹H NMR (400 MHz, Acetone) δ 0.91 (t, J=7.3 Hz, 3H). 1.25-1.07 (m, 5H), 1.67 (h, J=7.5 Hz, 2H), 2.68 (t, J=7.6 Hz, 2H), 2.85 (d, J=1.3 Hz, 1H), 3.48-3.37 (m, 1H), 3.69-3.59 (m, 1H), 4.22 (qt, J=7.3, 3.4 Hz, 2H), 4.69-4.46 (m, 4H), 6.29 (s, 1H), 7.25-7.15 (m, 2H), 7.37 (d, J=2.3 Hz, 1H), 7.45 (t, J=7.4 Hz, 1H), 7.54 (t, J=7.5 Hz, 2H), 7.82-7.64 (m, 5H), 7.87 (d, J=1.5 Hz, 4H), 8.00 (s, 1H).

### General Procedure for Saponification of O-alkylated N¹-Indazole Ester

### (Procedure E):

To a flask containing the ester (1 equiv) in THF: H₂O (2:1, v/v), LiOH (3.0 equiv) was added. The mixture was refluxed for 2 h. After reaction completion, monitored by TLC, the solution was concentrated in vacuo, diluted with water, and then the aqueous layer was acidified to pH 3 with 1 N HCl. The resulting white participate was filtered and dried under high vacuum to afford the target compound as a white solid.

Synthesis of 2-(5-(3-(4-([1,1′-biphenyl]-4-carbonyl)-2-propylphenoxy)propoxy)-1H-indazol-1-yl)acetic acid—Compound “MP-151”:

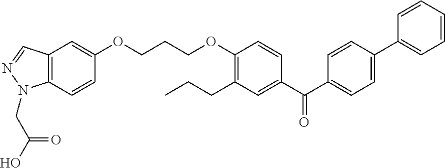 [SMILES: CCCc1cc(C(=O)c2ccc(-c3ccccc3)cc2)ccc1OCCCOc1ccc2c(cnn2CC(=O)O)c1]

To a solution of methyl 2-(5-(3-(4-([1,1′-biphenyl]-4-carbonyl)-2-propylphenoxy)propoxy)-1H-indazol-1-yl)acetate (0.1 g, 0.18 mmol) in THF (4 mL), was added LiOH (0.013 g, 0.54 mmol) solution in water (2 mL) then the reaction was performed according to procedure E to afford product as a white solid (0.08 g, 80% yield).

¹H NMR (400 MHz, DMSO-d6) δ 0.87 (t, J=7.6 Hz, 3 H), 1.56 (sext, J=7.6 Hz, 2H), 2.26 (quin, J=6.4 Hz, 2 H), 2.61 (t, J=7.6 Hz, 2 H), 4.23 (t, J=6.4 Hz, 2H), 4.28 (t, J=6.4 Hz, 2H), 5.14 (s, 2 H), 7.05 (dd, J=2.3, 8.7 Hz, 1H), 7.15 (d, J=8.7 Hz, 1H), 7.21 (d, J=2.3 Hz, 1H), 6.42-6.45 (m, 1H), 7.50-7.53 (m, 3H), 7.63-7.66 (m, 2H), 7.76-7.79 (m, 4H), 7.83-7.86 (m, 2H), 7.93 (s, 1H).

Synthesis of 2-(5-(3-(4-(4-fluorobenzoyl)-2-propylphenoxy)propoxy)-1H-indazol-1-yl)acetic acid—Compound “MP-154”:

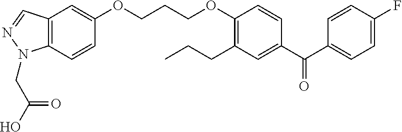 [SMILES: CCCc1cc(C(=O)c2ccc(F)cc2)ccc1OCCCOc1ccc2c(cnn2CC(=O)O)c1]

To a solution of methyl 2-(5-(3-(4-(4-fluorobenzoyl)-2-propylphenoxy)propoxy)-1H-indazol-1-yl)acetate (0.1 g, 0.19 mmol) in THF (4 mL), was added LiOH (0.014 g, 0.59 mmol) solution in water (2 mL) then the reaction was performed according to procedure E to afford product as a white solid (0.07 g, 78% yield).

¹H NMR (400 MHz, DMSO-d6) δ 0.87 (t, J=7.4 Hz, 3H), 1.52-1.58 (sext, J=7.4 Hz, 2H), 2.23-2.29 (m, 2H), 2.61 (t, J=7.4 Hz, 2 H), 4.20 (t, J=6.4 Hz, 2H), 4.28 (t, J=6.4 Hz, 2H), 4.94 (s, 2 H), 7.01 (dd, J=2.3, 8.7 Hz, 1H), 7.15 (d, J=8.7 Hz, 1H), 7.18 (d, J=2.3 Hz, 1H), 7.35-7.40 (m, 2H), 7.43 (d, J=8.7 Hz, 1H), 7.57-7.61 (m, 2H), 7.75-7.79 (m, 2H), 7.86 (s, 1H).

Synthesis of 2-(5-(3-(4-phenoxyphenoxy)propoxy)-1H-indazol-1-yl)acetic acid-compound “MH-103”:

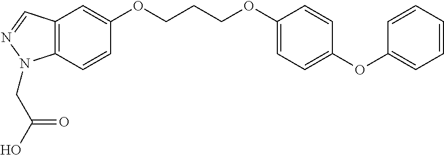 [SMILES: O=C(O)Cn1ncc2cc(OCCCOc3ccc(Oc4ccccc4)cc3)ccc21]

To a solution of methyl 2-(5-(3-(4-phenoxyphenoxy)propoxy)-1H-indazol-1-yl)acetate (0.1 g, 0.23 mmol) in THF (4 mL), was added LiOH (0.017 g, 0.69 mmol) solution in water (2 mL) then the reaction was performed according to procedure E to afford product as a white solid (0.09 g, 82%).

¹H NMR (400 MHz, DMSO) δ 2.21 (p, J=6.3 Hz, 2H), 4.16 (q, J=6.2 Hz, 4H), 5.22 (s, 2H), 6.92 (d, J=8.0 Hz, 2H), 6.95-7.04 (m, 4H), 7.04-7.11 (m, 2H), 7.23 (d, J=2.5 Hz, 1H), 7.34 (t, J=7.8 Hz, 2H), 7.55 (d, J=9.0 Hz, 1H), 7.95 (s, 1H), 13.03 (s, 1H).

Synthesis of 2-(5-(2-(9H-carbazol-9-yl)ethoxy)-1H-indazol-1-yl)acetic acid—compound “MH-107”:

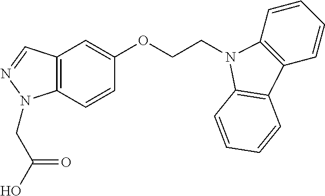 [SMILES: O=C(O)Cn1ncc2cc(OCCn3c4ccccc4c4ccccc43)ccc21]

To a solution of methyl 2-(5-(2-(9H-carbazol-9-yl)ethoxy)-1H-indazol-1-yl)acetate (0.1 g, 0.25 mmol) in THF (4 mL), was added LiOH (0.018 g, 0.75 mmol) solution in water (2 mL) then the reaction was performed according to procedure E to afford product as a white solid (0.07 g, 76%).

¹H NMR (400 MHz, DMSO) δ 4.38 (t, J=5.4 Hz, 2H), 4.84 (t, J=5.3 Hz, 2H), 4.95 (s, 2H), 6.81 (dd, J=9.0, 2.3 Hz, 1H), 7.12 (d, J=2.3 Hz, 1H), 7.22 (t, J=7.5 Hz, 2H), 7.39 (d, J=9.1 Hz, 1H), 7.48 (t, J=7.7 Hz, 2H), 7.72 (d, J=8.2 Hz, 2H), 7.83 (s, 1H), 8.16 (d, J=7.7 Hz, 2H).

Synthesis of 2-(5-(3-(4-([1.1′-biphenyl]-4-carbonyl)-2-propylphenoxy)propoxy)-1H-indazol-1-yl)-2-ethoxyacetic acid—compound “KV-180”:

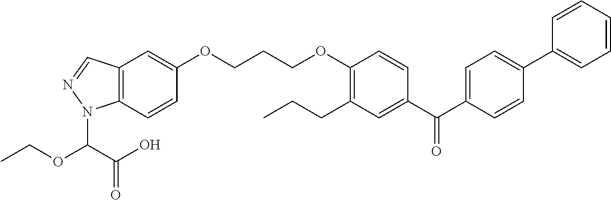 [SMILES: CCCc1cc(C(=O)c2ccc(-c3ccccc3)cc2)ccc1OCCCOc1ccc2c(cnn2C(OCC)C(=O)O)c1]

To a solution of ethyl 2-(5-(3-(4-([1,1′-biphenyl]-4-carbonyl)-2-propylphenoxy)propoxy)-1H-indazol-1-yl)-2-ethoxyacetate (0.1 g, 0.16 mmol) in THF (4 mL), was added LiOH (0.01 g, 0.48 mmol) solution in water (2 mL) then the reaction was performed according to procedure E to afford product as a white solid (0.08 g, 88% yield).

¹H NMR (400 MHz, Acetone) δ 0.94-0.90 (m, 3H), 1.13-1.09 (m, 3H), 1.67-1.58 (m, 2H), 2.34-2.35 (m, 2H), 3.66-3.58 (m, 1H), 3.43 — 3.37 (m, 2H), 3.63 (dd, J=9.4, 6.9 Hz, 1H), 4.32 (t, J=6.1 Hz, 2H), 4.38 (t, J=6.0 Hz, 2H), 6.28 (s, 1H), 7.08-7.18 (m, 2H), 7.29 (d, J=2.4 Hz, 1H), 7.38-7.55 (m, 3H), 7.61-7.80 (m, 5H), 7.84 (s, 4H), 7.96 (s, 1H).

Synthesis of 2-(5-(2-(4-([1,1′-biphenyl]-4-carbonyl)-2-propylphenoxy)ethoxy)-1H-indazol-1-yl)-2-ethoxyacetic acid—Compound “KV-196”:

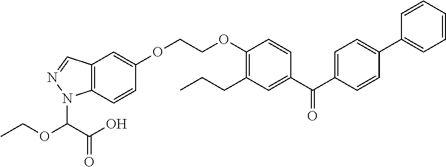 [SMILES: CCCc1cc(C(=O)c2ccc(-c3ccccc3)cc2)ccc1OCCOc1ccc2c(cnn2C(OCC)C(=O)O)c1]

To a solution of ethyl 2-(5-(2-(4-([1,1′-biphenyl]-4-carbonyl)-2-propylphenoxy)ethoxy)-1H-indazol-1-yl)-2-ethoxyacetate (0.1 g, 0.16 mmol) in THF (4 mL), was added LiOH (0.01 g, 0.49 mmol) solution in water (2 mL) then the reaction was performed according to procedure E to afford product as a white solid (0.08 g, 82% yield).

¹H NMR (400 MHz, Acetone) δ 0.77 (t, J=7.4 Hz, 3H), 1.00 (t, J=6.7, Hz, 3H), 2.54 (t, J=7.5 Hz, 2H), 3.28 (q, J=7.9 Hz, 2H), 3.51 (q, J=8.7 Hz, 2H), 4.42 (dd, J=13.9, 5.0 Hz, 4H), 6.18 (s, 1H), 6.96-7.10 (m, 3H), 7.24 (d, J=2.2 Hz, 1H), 7.31 (t, J=7.4 Hz, 1H), 740 (t, J=7.6 Hz, 2H), 7.52-7.62 (m, 3H), 7.65 (d, J=7.7 Hz, 2H), 7.72 (s, 3H), 7.87 (s, 1H).

Synthesis of 2-ethoxy-2-(5-(3-(4-(4-fluorobenzoyl)-2-propylphenoxy)propoxy)-1H-indazol-1-yl)acetic acid—compound “KV-160”:

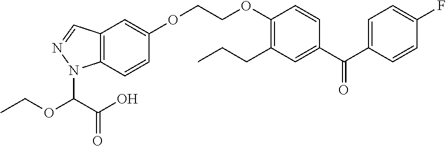 [SMILES: CCCc1cc(C(=O)c2ccc(F)cc2)ccc1OCCOc1ccc2c(cnn2C(OCC)C(=O)O)c1]

To a solution of methyl 2-ethoxy-2-(5-(3-(4-(4-fluorobenzoyl)-2-propylphenoxy)propoxy)-1H-indazol-1-yl)acetate (0.1 g, 0.18 mmol) in THF (4 mL), was added LiOH (0.01 g, 0.54 mmol) solution in water (2 mL) then the reaction was performed according to procedure E to afford product as a white solid (0.08 g, 80% yield).

¹H NMR (400 MHz, MeOD) δ 0.90 (t, J=7.3 Hz, 3H), 1.10 (t, J=7.0 Hz, 3H), 1.59 (q, J=7.5 Hz, 2H), 2.34 (p, J=6.0 Hz, 2H), 2.64 (t, J=7.6 Hz, 2H), 3.26 (t, J=8.2 Hz, 1H), 3.54 (dt, J=14.2, 6.9 Hz, 1H), 4.18-4.37 (m, 4H), 5.96 (s, 1H), 7.03-7.11 (m, 2H), 7.19-7.28 (m, 3H), 7.58-7.67 (m, 3H), 7.79 (dd, J=8.5, 5.5 Hz, 2H), 7.91 (s, 1H).

Synthesis of 2-ethoxy-2-(5-(2-(4-(4-fluorobenzoyl)-2-propylphenoxy)ethoxy)-1H-indazol-1-yl)acetic acid—compound “KV-192”:

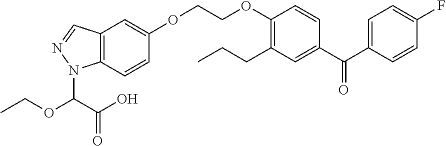 [SMILES: CCCc1cc(C(=O)c2ccc(F)cc2)ccc1OCCOc1ccc2c(cnn2C(OCC)C(=O)O)c1]

To a solution of ethyl 2-ethoxy-2-(5-(2-(4-(4-fluorobenzoyl)-2-propylphenoxy)ethoxy)-1H-indazol-1-yl)acetate (0.1 g, 0.19 mmol) in THF (4 mL), was added LiOH (0.01 g, 0.57 mmol) solution in water (2 mL) then the reaction was performed according to procedure E to afford product as a white solid (0.08, 81% yield).

¹H NMR (400 MHz, Acetone) δ 0.90 (t, J=8.0, 3H), 1.07-1.36 (m, 3H), 1.65 (h, J=7.4 Hz, 2H), 2.67 (t, J=7.6 Hz, 2H), 3.30-3.59 (m, 1H), 3.54-3.82 (m, 1H), 4.36-4.77 (m, 4H), 6.32 (d, J=1.3 Hz, 1H), 7.12-7.50 (m, 5H), 7.58-7.82 (m, 3H), 7.80-7.93 (m, 2H), 8.01 (s, 1H).

Scheme III: synthesis of methyl 2-(5-(3-(pyridine/substituted pyridine-2-ylamino)propoxy)-1H-indazol-1-yl)acetate

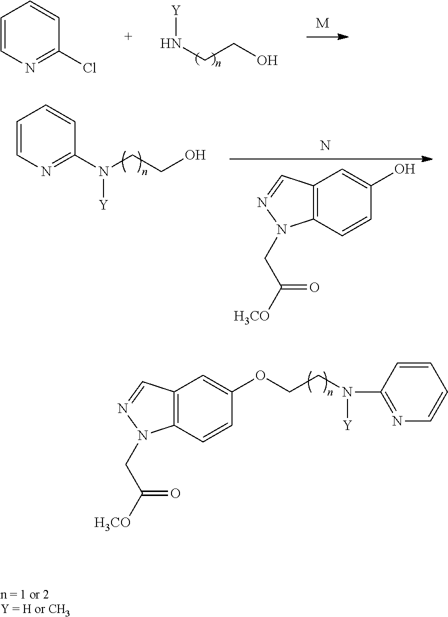

Reagents and conditions: M) 150° C., 16 h; N) PPh3, DEAD, nt, 16 h

Synthesis of 3-(pyridin-2-ylamino)propan-1-ol

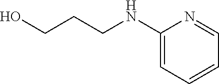 [SMILES: OCCCNc1ccccn1]

2-Chloropyridine (1 g, 8.8 mmol) was mixed with 3-aminopropan-1-ol (7.9 g, 105.0 mmol). The reaction mixture was heated at 150° C. for 16 h under a nitrogen atmosphere. After reaction completion, monitored by TLC, distilled water (20 mL) was added to the mixture. The mixture was extracted with EtOAc (3×30 mL). The organic layer was then dried over anhydrous sodium sulfate, then the solvent was evaporated under reduced pressure to give the crude product. The crude product was purified using flash chromatography using DCM: MeOH (gradient 0-10% MeOH) to give the target compound as a white crystal (1.04 g, 78% yield). ¹14 NMR (400 MHz, CDCl₃) δ 1.75 (p, J=6.0 Hz, 2H), 3.53 (q, J=6.1 Hz, 2H), 3.64 (t, J=5.6 Hz, 2H), 3.96 (s, 1H), 4.78 (s, 1H), 6.40 (d, J=8.4 Hz, 1H), 6.54 (ddd, J=7.0, 5.3, 1.1 Hz, 1H), 7.38 (ddd, J=8.7, 7.0, 1.7 Hz, 1H), 7.89-8.07 (m, 1H).

Synthesis of methyl 2-(5-(3-(pyridin-2-ylamino)propoxy)-1H-indazol-1-yl)acetate

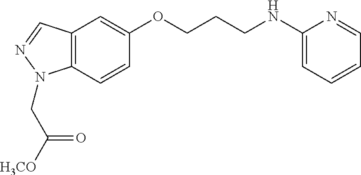 [SMILES: COC(=O)Cn1ncc2cc(OCCCNc3ccccn3)ccc21]

To a mixture of methyl 2-(5-hydroxy-1H-indazol-1-yl) acetate (0.1 g, 0.48 mmol), and 3-(pyridin-2-ylamino)propan-l-ol (0.07 g, 0.48 mmol)in anhydrous THF (5 mL), was added triphenylphosphine (0.16 g, 0.63 mmol) and cooled to 0° C. for 10 minutes. A solution of DEAD (0.11 g, 0.63 mmol) in anhydrous THF (2 mL) was added dropwise to the reaction mixture and allowed to stir at room temperature over night under N₂ atmosphere. After reaction completion, monitored by TLC, distilled water (5 mL) was added to the reaction mixture. The mixture was extracted with EtOAc (2×20 mL). The organic layer was then dried over anhydrous sodium sulfate, then the solvent was evaporated under reduced pressure to give the crude product. The crude product was purified using flash chromatography over silica gel eluting with hexane: EtOAc (gradient 0-40% EtOAc) to give compound (0.03 g, 18% yield) as a colorless oil.

¹H NMR (400 MHz, CDCl₃) δ 1.41 (t, J=7.1 Hz, 2H), 1.71 (m, 2H) 3.78 (s, 2H), 4.36-4.23 (m, 4H), 5.18 (s, Hz, 2H), 7.24-7.36 (m, 2H), 7.44-7.62 (m, 5H), 8.38 (br s, 1H).

### Example 2

### Cell-Based PPAR Transactivation Assay

The assay was performed using cell-based human PPAR nuclear receptor reporter assay kits (NR1C1 for PPARα; NR1C2 for PPARδ/β, and NR1C3 for PPARγ, Indigo Biosciences) by following the manufacturer's instruction. In brief, PPAR reporter cells were added to CRM medium (Indigo Biosciences), seeded into wells of a 96-well culture plate and incubated for 5 hours in a cell culture incubator at 37° C. with 5% CO₂. The medium was then removed, and the cells were treated with test compounds at different concentrations in CSM medium (Indigo Biosciences) for 24 h inside the incubator. Reference compounds (Fenofibrate from Sigma or GW590735 for PPARα; GW0742 for PPARβ/δ; and rosiglitazone for PPARγ from the kits, Indigo Biosciences, Inc.), were tested simultaneously as positive controls. DMSO (Sigma) at 0.1% (v/v) was used as negative baseline control (C). Each treatment was tested in duplicate wells. At the end point of the experiments, the treatment medium was discarded and Luciferase Detection Reagent (Indigo Biosciences) was added. Following a 10-minute rest period, the intensity of light emission (in units of “Relative Light Units; RLU”) from each assay well was quantified using a plate-reading luminometer (Synergy H1 plate reader, BioTek). In addition, live cell multiplex assay (Indigo Biosciences) was combined with the activation assays to detect potential cytotoxic effect, if any, of the test chemicals to normalize the signals as per manufacturer's instruction.

### Cellular PPAR Transactivation Results

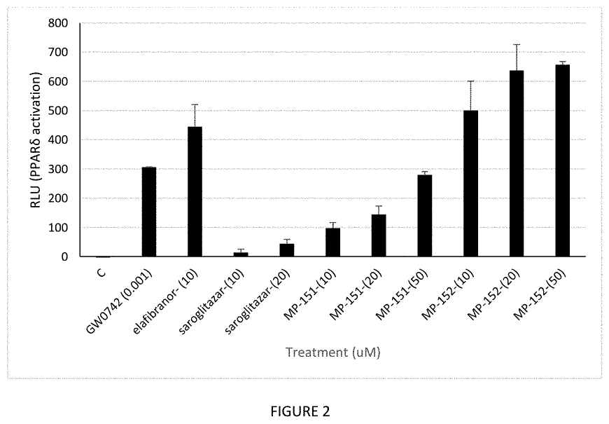

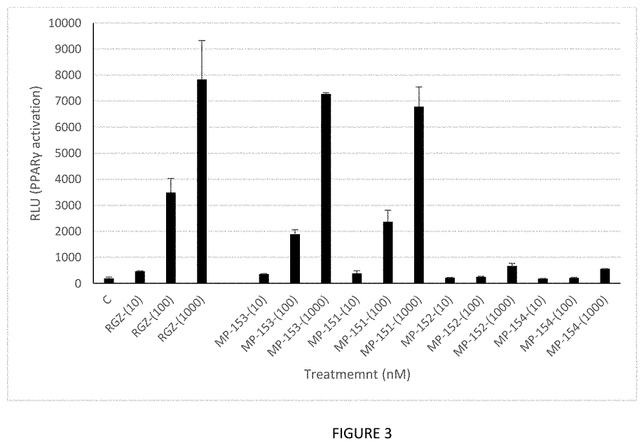

In the PPARα transactivation assay (FIG. 1), MP-151 and MP-153 were found to be active at 5 μM while MP-152 and MP-154 were active starting at 1 μM. The reference compound fenofibrate was not active at the test range (1-10 μM) while PPARα agonist GW590375 was active starting at 0.001 μM. In PPARδ/β transactivation assay (FIG. 2), none of the test compounds (MP-151, MP-152, MP-153 and MP-154) were active up to 1 μM (data not shown). However, MP-151 and MP-152 activated PPARδ/β at higher concentrations in the range of 10-50 μM. The reference PPARδ/β agonist GW0742 was active at 0.001 μM. Known PPAR agonists, elafibranor and saroglitazar, also showed activities at tested concentrations. In PPARγ transactivation assay (FIG. 3) MP-151, MP-153 and rosiglitazone are active starting at 10-100 nM, while MP-152 and MP-154 are active at 1,000 nM. The results of PPAR transactivation assay showed that MP-151, MP-152, MP-153 and MP-154, among other tested chemicals, are PPAR agonists on PPARα and/or PPARδ/β and/or PPARγ activation.

It was observed that KV-157, KV-160, KV-163A, KV-165, KV-180, KV-192, KV-194A, and KV 196 are not effective on activating PPARα (at 10 μM) but are highly effective on activating PPARγ (at 1 μM), except for KV-160 and KV-192, which activated PPARγ at 10 μM.

### Example 3

### Cytotoxicity in Human Epidermal Keratinocyte (HaCaT) Cells

Cytotoxicity of test compounds were tested using immortal human epidermal keratinocyte cell culture (HaCaT, purchased from AddexBio). The cell culture was maintained in DMEM high glucose (4.5g/L) medium supplemented with 10% fetal bovine serum (FBS) and 1% (v/v) penicillin/streptomycin solution (Invitrogen, Thermo Fisher) and kept in an incubator at 37° C. with 5% CO₂. Cell viability was determined by MTT assay ((Twentyman & Luscombe, Can Nurse. 2012 May;108(5):22-7). Briefly, HaCaT cells were seeded at 5×10³ cells/well in 100 μl medium in 96-well plates and incubated overnight (˜18 h). After removing the medium, the cells were then incubated with a series of concentrations of the compounds in fresh medium for 72 hours in the incubator. At the end point of the experiments, 10 μl of MTT stock solution (5 mg/mL in PBS) was added to each well and mixed with medium by gentle tapping. The cells were further incubated for 3 hours in the incubator. The reaction was stopped by removing supernatant and adding 100 μl of DMSO to each well in 96-well plate in order to completely dissolve the formazan formed. The absorbance was measured at 540 nm using a Synergy H1 plate reader (BioTek). Cytotoxicity is calculated as a percentage of the treated wells over the non-drug treated control wells based on the absorbance values. There were triplicate wells for each treatment and the experiment was independently repeated at least three times. The data are presented as the mean and standard derivation of the experiments.

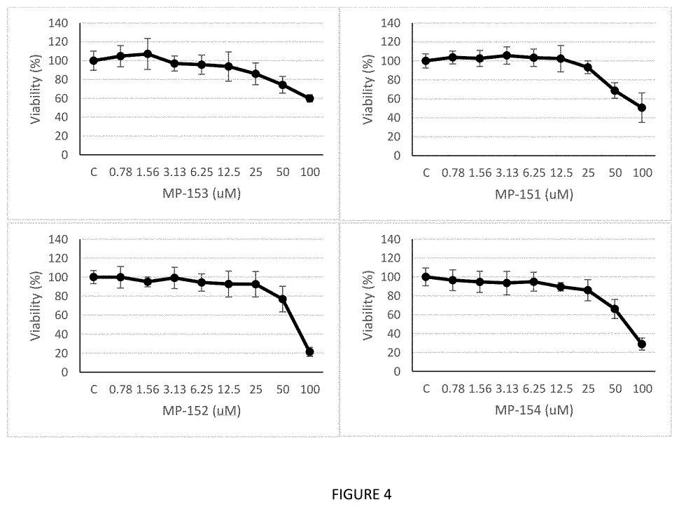

Cytotoxicity in HaCaT cells results—In the cytotoxicity assay, all the test compounds (MP-151, MP-152, MP-153 and MP-154) were found non-cytotoxic to HaCaT cells up to 25 μM under the test conditions (FIG. 4), which is 5 to 250 times higher than the concentration required to activate PPAR receptors.

### Example 4

### Anti-Inflammatory Effect in Three (3) HaCaT Cell-Based Models

Three in vitro models based on HaCaT cell culture were tested. HaCaT cells were maintained as described above in DMEM high glucose (4.5g/L) medium supplemented with 10% FBS and 1% (v/v) penicillin/streptomycin solution (Invitrogen, Thermo Fisher) and kept in an incubator at 37° C. with 5% CO₂. For experiments, the cells were seeded at 5×10⁴ cells/well in 1 mL of medium to a 24-well culture plate (Nunc, Thermo Fisher) and incubated overnight.

(1) For the imiquimod (IMQ)-stimulated HaCaT model, IMQ (Alfa Aesar) at 50 μM final concentration was used in the experiments; (2) for TNFα/IL-17A-stimulated-HaCaT model, 2 ng/mL of each cytokine (R&D Systems) was used; and (3) for the lipopolysaccharide (LPS, Sigma) simulated HaCaT cell model, 20 μg/mL LPS was used. For drug chemical treatment the old medium in the culture plates described above was discarded and the corresponding stimulant (IMQ, TNFα/IL-17A, or LPS) with or without test chemicals in 0.5 mL fresh medium was added to each cell well. There were duplicate wells for each treatment. The plate was incubated for another 24 h and the supernatant was collected for cytokine analysis, namely IL-6 and IL-8, using the corresponding ELISA kits (Thermo Fisher). The experiments were repeated independently to confirm the observation.

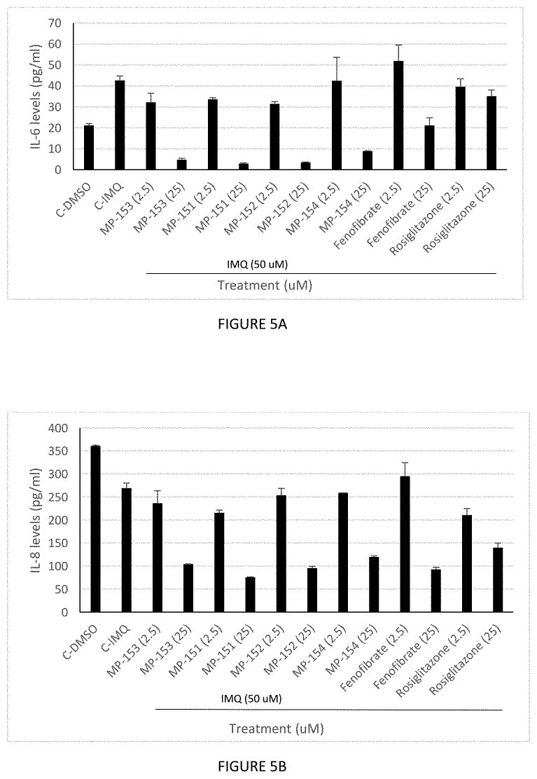

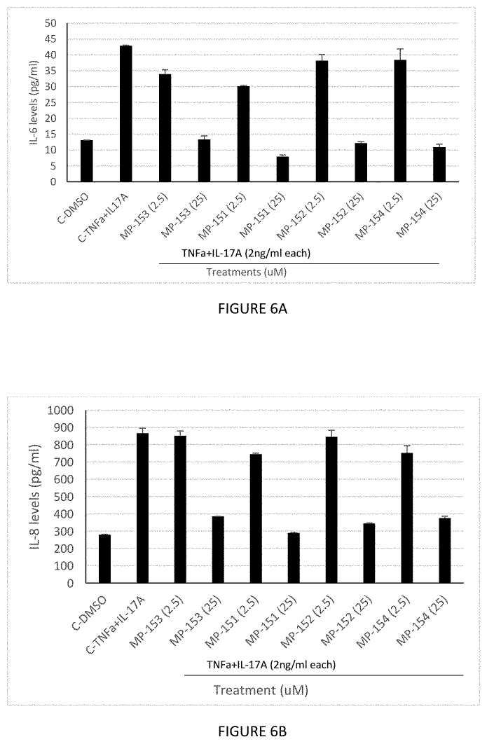

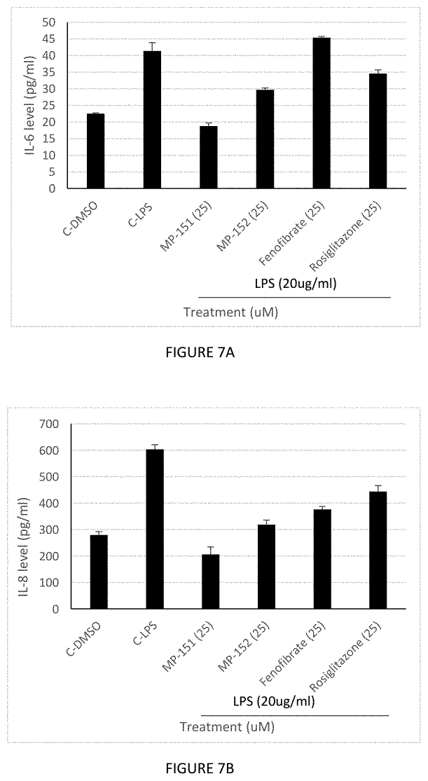

In vitro anti-inflammatory effect in three HaCaT cell-based models results—In the IMQ-stimulated HaCaT cell model (FIGS. 5A and 5B), the test compounds (MP-151, MP-152 and MP-153) significantly inhibited production of IL-6 by HaCaT cells at 2.5 and 25 μM when compared to control. Whereas, MP-154 had no anti-inflammatory effect at 2.5 μM, but significantly inhibited production of IL-6 at 25 μM. The reference compounds rosiglitazone (a PPARγ agonist) and fenofibrate (a PPARα agonist), are relatively less active than test compounds. When tested for IL-8 production, all the test compounds significantly inhibited IL-8 production at 25 μM and had no effect at 2.5 μM concentration, except for MP-151 and rosiglitazone (FIG. 5B). In the TNFα/IL-17A -stimulated HaCaT cell model (FIGS. 6A and 6B), all the test compounds showed significant inhibition on the production of IL-6 and IL-8 at 25 μM while less active or inactive at 2.5 μM. In the LPS stimulated HaCaT cell model (FIGS. 7A and 7B), MP-151, MP-152 and rosiglitazone reduced the production of IL-6 at 25 uM while fenofibrate was not effective at this concentration (FIG. 7A). When tested for IL-8 production, all the test compounds reduced the production of IL-8 at 25 μM, and MP-151 and MP-152 are more active than rosiglitazone and fenofibrate (FIG. 7B).

The abovementioned results indicate that the PPAR agonist(s) can be potentially used as anti-inflammatory therapeutics. It should be noted that anti-inflammatory effect of test compounds is not due to cytotoxicity effect as demonstrated in the cytotoxicity assays (FIG. 4).

### Example 5

### Anti-Inflammatory Effect of Combined Test Compounds (Abn-CBD/CBD and PPAR Agonists)

HaCaT cells were maintained as described above in DMEM high glucose (4.5 g/L) medium supplemented with 10% FBS and 1% (v/v) penicillin/streptomycin solution (Invitrogen, Thermo Fisher) and kept in an incubator at 37° C. with 5% CO₂. For experiments the cells were seeded at 5×10⁴ cells/well in 0.5 mL of medium to a 24-well culture plate (Nunc, Thermo Fisher) and incubated overnight. The old medium in the culture plates was discarded and the corresponding stimulant, TNFα/IL-17A (R&D Systems) at 2 ng/mL each, with or without test chemicals in 0.5 mL of fresh medium was added to each cell well in the plate. There were duplicate wells for each treatment. The plate was incubated for another 24 h and the supernatant was collected for cytokine analysis, namely IL-6 and IL-8, using ELISA kits (Thermo Fisher).

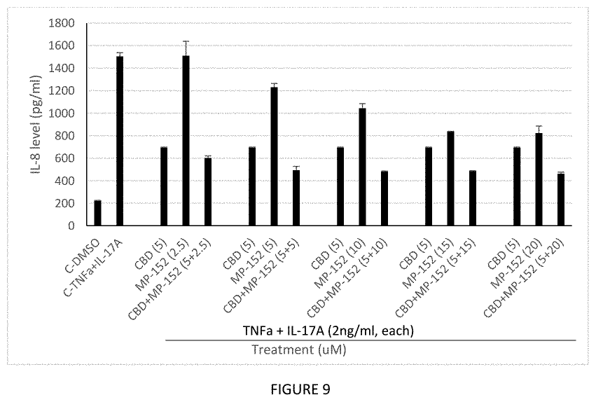

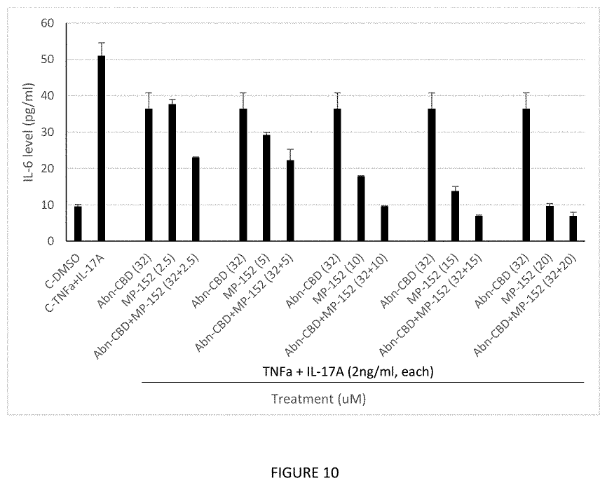

Anti-inflammatory effect of combined test compounds (Abn-CBD/CBD and PPAR agonists) results—Initial studies in the range 1 to 30 μM showed that CBD reduced the production of both IL-8 and IL-6 in HaCaT model. The optimum range of CBD for cytokine inhibition was found to be from 1 to 10 μM. At higher concentrations, e.g. 30 μM, CBD was found to inhibit HaCaT growth and could target hyperproliferation in psoriasis. In studies of combinatory effect the test compounds, MP-151 and MP-152, when combined with either Abn-CBD or CBD, significantly inhibited the production of inflammatory cytokines, IL-6 and IL-8, by HaCaT cells, compared to individual compound treatment, indicating a potential synergistic effect between cannabinoids and PPAR agonists on anti-inflammation (FIGS. 8-10). The combination of CBD (5 μM) and PPAR agonist MP-151 (5 or 10 μM) significantly inhibited IL-6 production compared to individual treatment alone (FIG. 8A). Similarly, the combination of CBD (5 μM) with MP-151 (5-20 μM) significantly reduced the IL-8 levels compared to individual treatment alone (FIG. 8B). Combination of CBD (5 μM) and PPAR agonist MP-152 (2.5-20 μM) provided better efficacy than individual treatments on inhibiting inflammatory cytokine IL-8 production as shown in FIG. 9. Combination of Abn-CBD (32 μM) and PPAR agonist MP-152 (2.5-20 μM) provided better efficacy than individual treatments on inhibiting inflammatory cytokine IL-6 production as shown in FIG. 10.

### Example 6

### Dose-Response Studies in Mice

### Materials and Methods

The animal experiment is conducted in accordance with the Guidelines for the Institutional Animal Care and Use Committee. Both male and female BALB/c mice from Rodenta Bioserve, India, at 7-8 weeks of age and 20-25 gram of body weight are housed in a laboratory environment with constant temperature (24.0±1.0° C.) and relative humidity (45.0-55.0%) on a 12 h light/dark cycles. These animals are provided free access to unlimited amounts of food and water during the entire experiment. The animals are divided into 12 groups of 8 mice each and shaved on the back on day 0 for an area of 2×2 cm . Human-like psoriasis in mice is induced by topical application of 5% Imiquimod (IMQ) procured from AK Scientific, USA, and formulated in the base cream at desired percentages in-house, in shaved area and right ear (0.5×0.5 cm). IMQ or equal amount of vehicle cream (Versapro™, Medisca) is topically applied on both the shaved back (62.5 mg of 5% IMQ cream) and the right ear (16 mg of 5% IMQ cream) of mice, once daily for 16 consecutive days (day 1 to day 16). Vehicle, clobetasol propionate (CP, Sigma. 0.05% w/w; positive control), test creams, and CBD (Ilesol Pharmaceuticals), respectively, is applied topically on both the shaved back (80 mg cream) and the right ear (20 mg cream) of mice, once daily from day 10 to day 16. The study ends at day 17. At stated time points the severity of psoriasis is evaluated using the PASI score (Psoriasis Area Severity Index: sum of erythema, scaling and thickening, scored independently from 0 to 4 for each mouse; max PASI score=12). Tissue and blood samples are collected for histopathology, biochemistry and immunoassay from each animal at various points.

### Results

Based on one way ANOVA, there was no significant changes in the body weight of the animals among treatment groups when compared with IMQ control (data not shown).

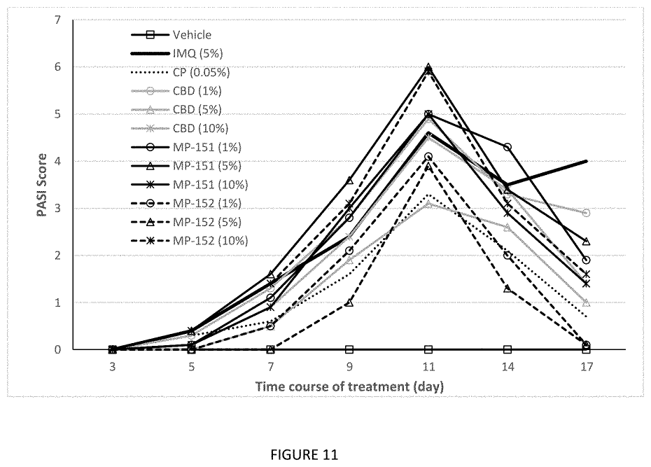

All three test compounds, MP-151, MP-152 and CBD, were well tolerated by the mice for the seven-day consecutive treatment even at 10% creams. No visual damage on the skin was noticed. The PASI score reached peak level at day 11 and declined thereafter after drug treatment. Note that the PASI score in IMQ group was 4 till day 17. (FIG. 11). The positive control (CP) was effective in reducing the PASI score as expected. Among the treatment groups, MP-152 at 1% and 5% were the most active and were even more effective than CP in reducing the PASI score, indicating the potential of the compounds of the present disclosure as effective therapeutics in inflammatory diseases, such as psoriasis.

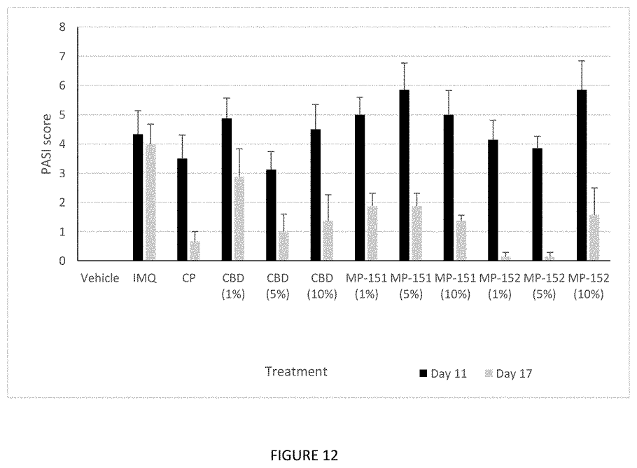

When comparing PASI scores on day 11, the peak level, and day 17, the end point of the study, all treatment groups, except for 1% CBD, significantly reduced PASI score after 7-day treatment (FIG. 12).

1. Manley P W, Cowan-Jacob S W, Mestan J. Advances in the structural biology, design and clinical development of Bcr-Ab1 kinase inhibitors for the treatment of chronic myeloid leukaemia. Biochim Biophys Acta. 2005 Dec 30;1754(1-2):3-13. doi: 10.1016/j.bbapap.2005.07.040. Epub 2005 Sep. 8. PMID: 16172030.

2. Chousterman B G, Swirski F K, Weber G F. Cytokine storm and sepsis disease pathogenesis. Semin Immunopathol. 2017 July;39(5):517-528. doi: 10.1007/s00281-017-0639-8. Epub 2017 May 29. PMID: 28555385.

3. Miller G E, Chen E, Parker K J. Psychological stress in childhood and susceptibility to the chronic diseases of aging: moving toward a model of behavioral and biological mechanisms. Psychol Bull. 2011 November;137(6):959-97. doi: 10.1037/a0024768. PMID: 21787044; PMCID: PMC3202072.

4. Furman D, Campisi J, Verdin E, Carrera-Bastos P, Targ S, Franceschi C, Ferrucci L, Gilroy D W, Fasano A, Miller G W, Miller A H, Mantovani A, Weyand C M, Barzilai N, Goronzy J J, Rando T A, Effros R B, Lucia A, Kleinstreuer N, Slavich G M. Chronic inflammation in the etiology of disease across the life span. Nat Med. 2019 December;25(12):1822-1832. doi: 10.1038/s41591-019-0675-0. Epub 2019 Dec. 5. PMID: 31806905; PMCID: PMC7147972.

5. Greten F R, Grivennikov S I. Inflammation and Cancer: Triggers, Mechanisms, and Consequences. Immunity. 2019 Jul 16;51(1):27-41. doi: 10.1016/j.immuni.2019.06.025. PMID: 31315034; PMCID: PMC6831096.

6. Zumsteg A, Christofori G. Corrupt policemen: inflammatory cells promote tumor angiogenesis. Curr Opin Oncol. 2009 January;21(1):60-70. doi: 10.1097/CCO.0b013e32831bed7e. PMID: 19125020.

7. Zamarron B F, Chen W. Dual roles of immune cells and their factors in cancer development and progression. Int J Biol Sci. 2011;7(5):651-8. doi: 10.7150/ijbs.7.651. Epub 2011 May 21. PMID: 21647333; PMCID: PMC3107473.

8. Fuentes E, Guzmán-Jofre L, Moore-Carrasco R, Palomo I. Role of PPARs in inflammatory processes associated with metabolic syndrome (Review). Mol Med Rep. 2013 December;8(6):1611-6. doi: 10.3892/mmr.2013.1714. Epub 2013 Oct. 7. PMID: 24100795.

9. Prussick R, Prussick L, Nussbaum D. Nonalcoholic Fatty liver disease and psoriasis: what a dermatologist needs to know. J Clin Aesthet Dermatol. 2015 March;8(3):43-5. PMID: 25852814; PMCID: PMC4382145.

10. Nestle, F. O., D. H. Kaplan, and J. Barker, Psoriasis. New England Journal of Medicine, 2009. 361(5): p. 496-509.

11. Laws, P. M. and H. S. Young, Topical treatment of psoriasis. Expert Opin Pharmacother, 2010. 11(12): p. 1999-2009.

12. Saraceno, R., et al., Calcipotriene/betamethasone in the treatment of psoriasis: a review article. Expert Opin Pharmacother, 2009. 10(14): p. 2357-65.

13. Murphy, G. and K. Reich, In touch with psoriasis: topical treatments and current guidelines. J Eur Acad Dermatol Venereol, 2011. 25 Suppl 4: p. 3-8.

14. Williams, H. C., Clinical practice. Atopic dermatitis. N Engl J Med, 2005. 352(22): p. 2314-24.

15. Habif, T., A Color Guide to Diagnosis and Therapy (Expert Consult—Online and Print. 5th ed. Clinical Dermatology 2010: Elsevier. 1040.

16. Rahman, M., et al., Classical to current approach for treatment of psoriasis: a review. Endocr Metab Immune Disord Drug Targets, 2012. 12(3): p. 287-302.

17. Castela, E., et al., Topical corticosteroids in plaque psoriasis: a systematic review of risk of adrenal axis suppression and skin atrophy. J Eur Acad Dermatol Venereol, 2012. 26 Suppl 3: p. 47-51.

18. Reich, K. and A. Bewley, What is new in topical therapy for psoriasis? J Eur Acad Dermatol Venereol, 2011. 25 Suppl 4: p. 15-20.

19. Repetto, M., Lopez-Artiguez, M., Martinez, D., Separation of cannabinoids. United Nations Office on Drugs and Crime (UNODC), 1976.

20. Narayanaswami K., G. H. C., Bam H. l., Stability of Cannabis sativa L. samples and their extracts, on prolonged storage in Delhi. United Nations Office on Drugs and Crime (UNODC), 1978.

21. Smith, R. N. and C. G. Vaughan, The decomposition of acidic and neutral cannabinoids in organic solvents. Journal of Pharmacy and Pharmacology, 1977. 29(1): p. 286-290.

22. U.S. Pat. No. 7,700,368B2, Methods of purifying cannabinoids from plant material, U.S. Patent, Editor.

23. Aubin, A., Purification of Cannabidiol from Hemp Oil Using the Prep150 LC System. Water Application Notes., 2015. Library Number: APNT134830443; Part Number: 720005287EN.

24. Remington, J. P. and A. R. Gennaro, Remington's pharmaceutical sciences. 1990, Easton, Pa.: Mack Pub. Co.

25. Reynolds, J. E. F. and W. Martindale, Martindale: the extra pharmacopoeia. 1993, London: Pharmaceutical P.

26. McEwen, G. N. W., John A & Cosmetic, Toiletry and Fragrance Association, International cosmetic ingredient handbook. 3rd ed, ed. G. N. M. John A. Wenninger, Jr. 1992, Washington, D.C.

27. Anthony L. L. Hunting (ed.), A Formulary of Cosmetic Preparations (Vol. 2)—Creams, Lotions and Milks,”. (Ed.). ISBN 10: 187022809X ISBN 13: 9781870228091. Publisher: Micelle Pr, 1993.

28. Miwa and Spencer, Proc. Second Int. Conf. on Jojoba and Uses, Ensenada, Baja Calif, Mexico, 229-243, 1976.

29. Twentyman P R, Luscombe M., A study of some variables in a tetrazolium dye (MTT) based assay for cell growth and chemosensitivity, 1987. Br. J. Cancer, vol. 56, pp. 279-285.

All citations are hereby incorporated by reference.

The present invention has been described with regard to one or more embodiments. However, it will be apparent to persons skilled in the art that a number of variations and modifications can be made without departing from the scope of the invention as defined in the claims.

1-48. (canceled)

49. A compound having the formula: 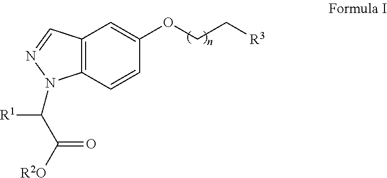
wherein:
n is 1 or 2;
R¹ is H, OC₂H₅, or O-alkyl;
R² is H, CH₃, or an alkyl chain; and
R³ is cycloalkyl, cycloalkylene, phenyl, substituted phenyl, phenoxyl, substituted phenoxyl, heterocyclyl, substituted heterocyclyl, amine, or substituted amine.

50. The compound of claim 49, or a pharmaceutically acceptable salt, stereoisomer, or solvate thereof, wherein:
n is 1 or 2;
R¹ is H;
R² is H or CH₃; and
R³ is:

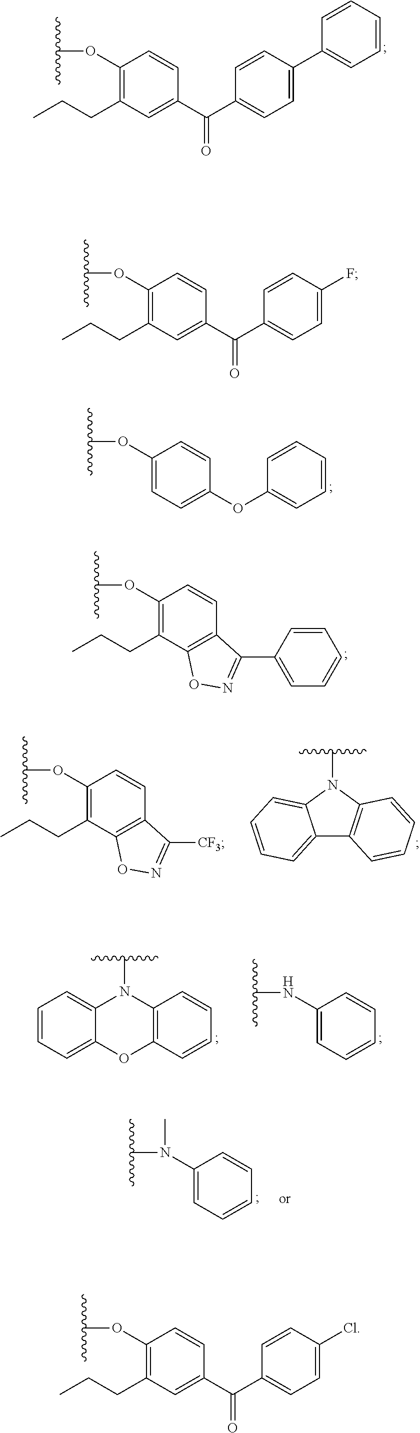

51. The compound of claim 49, or a pharmaceutically acceptable salt, stereoisomer, or solvate thereof, wherein:
n is 1 or 2;
R¹ is OC₂H₅;
R² is H or CH₃; and
R³ is:

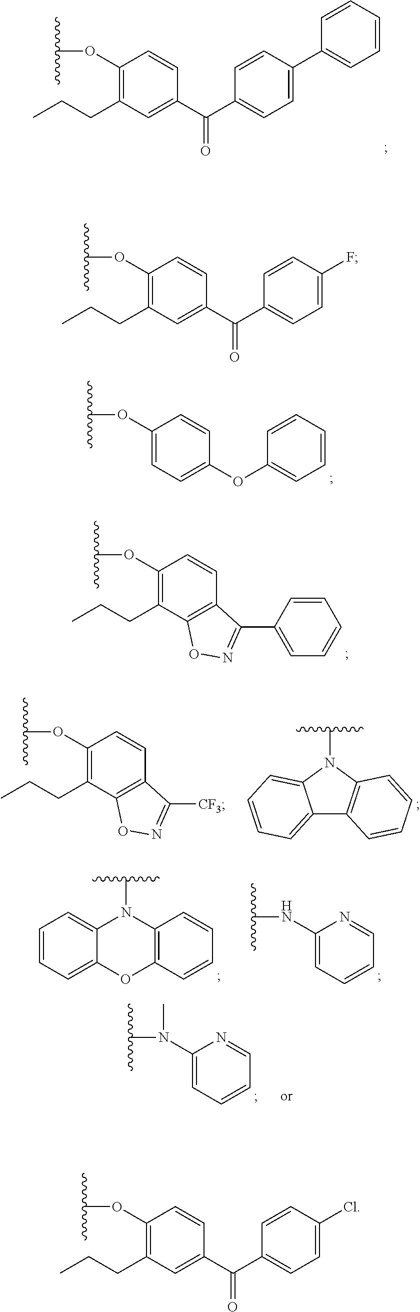

52. The compound of claim 49, or a pharmaceutically acceptable salt, stereoisomer, or solvate thereof, wherein:
n is 1 or 2;
R¹ is H or OC₂H₅;
R² is CH₃; and
R³ is:

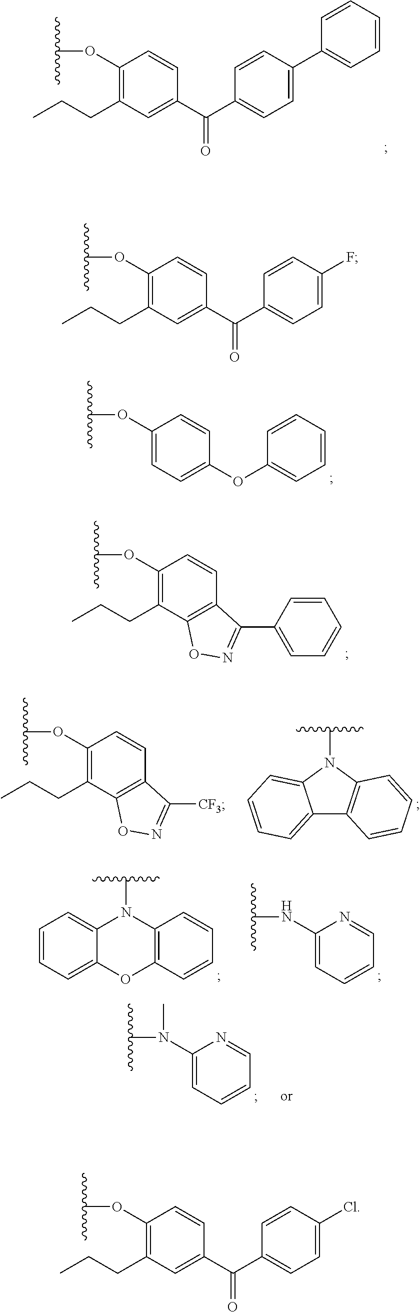

53. The compound of claim 49, or a pharmaceutically acceptable salt, stereoisomer, or solvate thereof, wherein:
n is 1 or 2;
R¹ is H or OC₂H₅;
R² is H; and
R³ is:

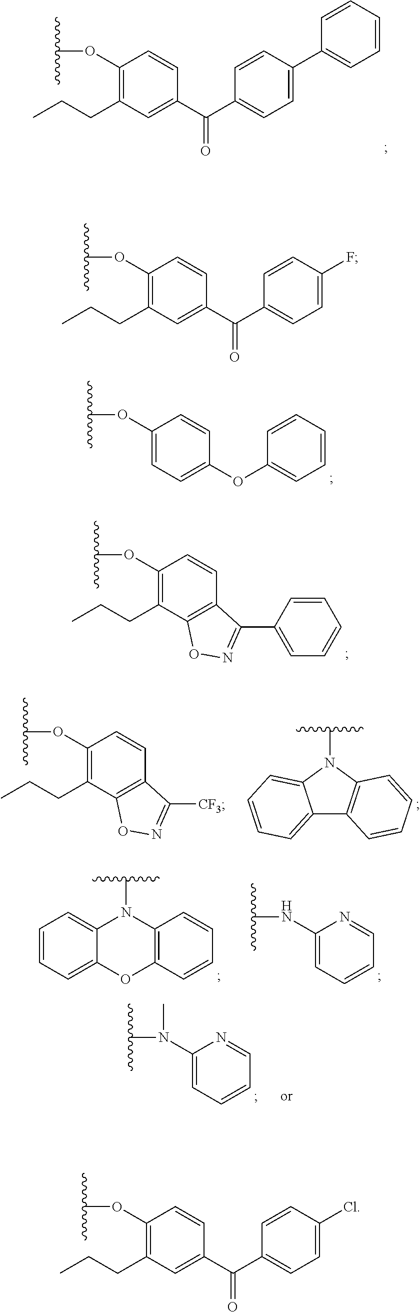

54. The compound of claim 49, wherein n is 1 or 2.

55. The compound of claim 49, selected from the group consisting of: 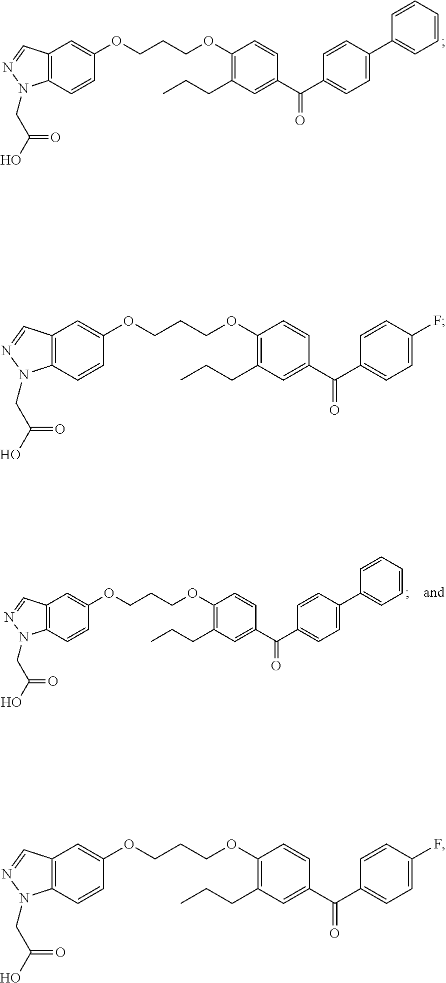 [SMILES: CCCc1cc(C(=O)c2ccc(-c3ccccc3)cc2)ccc1OCCCOc1ccc2c(cnn2CC(=O)O)c1 ; CCCc1cc(C(=O)c2ccc(F)cc2)ccc1OCCCOc1ccc2c(cnn2CC(=O)O)c1 ; CCCc1cc(C(=O)c2ccc(-c3ccccc3)cc2)ccc1OCCCOc1ccc2c(cnn2CC(=O)O)c1 ; CCCc1cc(C(=O)c2ccc(F)cc2)ccc1OCCCOc1ccc2c(cnn2CC(=O)O)c1]
or a pharmaceutically acceptable salt, stereoisomer, or solvate thereof.

56. A pharmaceutical composition comprising the compound of claim 49 and one or more pharmaceutically acceptable excipients.

57. The pharmaceutical composition of claim 56, further comprising at least one cannabinoid.

58. The pharmaceutical composition of claim 57, wherein the cannabinoid comprises cannabidiol (CBD) or Abnormal Cannabidiol (Abn-CBD).

59. The pharmaceutical composition of claim 56, comprising at least 0.0001% by weight of the compound of claim 49.

60. A method for treating a PPAR mediated disorder or condition in a subject, the method comprising administering the compound of claim 49.

61. The method of claim 60, wherein the PPAR mediated disorder or condition comprises skin disorders, peripheral diseases, steatohepatitis, type 2 diabetes, Alzheimer's disease, Parkinson's disease, multiple sclerosis, diabetes, metabolic syndromes, hyperlipemia, high-blood pressure, vascular disorders, dermatitis, psoriasis, inflammation, hepatitis, fatty liver, liver fibrosis, NASH (non-alcoholic steatohepatitis), obesity, liver cancer, cirrhosis, primary biliary cirrhosis, viral hepatitis, hepatic fibrosis, insulin resistance, impaired glucose tolerance, hyperinsulinemia, hypertriglyceridemia, non-alcoholic fatty liver disease, type 1 diabetes, hypercholesterolemia, nephropathy, pancreatitis, nephritis, stroke, arteriosclerosis, atherosclerosis, coronary heart disease, eczema, impaired wound healing, asthma, Crohn's disease, inflammatory bowel syndrome, ophthalmic inflammation, rheumatoid arthritis, and/or neurodegenerative disorders.

62. The method of claim 61, wherein the skin disorder comprises an inflammatory and/or autoimmune skin disorder.

63. The method of claim 62, wherein the inflammatory and/or autoimmune skin disorder is psoriasis, psoriatic arthritis, atopic dermatitis, inflammation, eczema, or dermatitis.

64. The method of claim 62, wherein the skin disorder comprises generalized topical skin wounds and wherein the treatment comprises wound healing of the topical skin wounds.

65. The method of claim 62, wherein the treating comprises an extended treatment period or a protracted treatment period.

66. A combination of a peroxisome proliferator-activated receptor (PPAR) agonist and a cannabinoid, for use in treating a PPAR mediated disorder or condition.

67. The combination of claim 66, wherein the PPAR agonist is the compound of claim 49.

68. The combination of claim 66, wherein the cannabinoid is cannabidiol (CBD) or Abnormal Cannabidiol (Abn-CBD).
